(19) United States
(12) Patent Application Publication
(10) Pub. No.: US 2008/0319227 A1
(43) Pub. Date: Dec. 25, 2008

(54) Processes for the Preparation of Modafinil and Analogs Thereof

(76) Inventors: Sidney Liang, Olivette, MO (US); John R. Duchek, St. Louis, MO (US); Carl J. Schaefer, Crestwood, MO (US)

Correspondence Address: Mallinckrodt Inc., 675 McDonnell Boulevard, HAZELWOOD, MO, 63042, US

(21) Appl. No.: 12/096,624

(22) PCT Filed: Nov. 27, 2006
(86) PCT No.: PCT/US2006/045431

§ 371 Date: Jun. 9, 2008

Related U.S. Application Data
(60) Provisional application No. 60/749,126, filed on Dec. 9, 2005.

(51) Int. Cl. C07C 315/02

(52) U.S. Cl. 564/162

(57) ABSTRACT

The present invention generally relates to an improved process for preparing modafinil and analogs thereof. The process minimizes impurities and improves the overall yield by oxidizing a modafinil intermediate compound in a reaction mixture including an alcohol and an organic acid at a ratio of from about 1:1 to about 80:1 (by volume).

### FIELD OF THE INVENTION

The present invention generally relates to improved processes for preparing modafinil and analogs thereof. More specifically, the processes include oxidizing a modafinil intermediate compound in a reaction mixture including an alcohol and an organic acid.

### BACKGROUND OF THE INVENTION

Modafinil, also known as benzhydrylsulfinylacetamide or 2-[(diphenyl)sulfinyl]acetamide, corresponds to the structure:

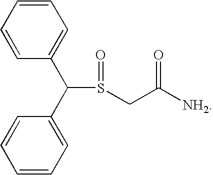

Modafinil is a synthetic acetamide derivative that exerts a wakefulness-promoting effect. Modafinil has been approved by the United States Food and Drug Administration for use in the treatment of excessive daytime sleepiness associated with narcolepsy.

Synthetic preparations of modafinil and similar compounds were first disclosed by Lafon in U.S. Pat. No. 4,177,290 ('290). The compounds were described as having useful pharmaceutical activity on the central nervous system. In Example 1 of '290, modafinil was prepared by reacting benzhydrylthioacetic acid with thionyl chloride to produce benzhydrylthioacetyl chloride. The chloride was then converted to benzhydrylthioacetamide by reaction with ammonia in methylene chloride. The sulfide atom of benzhydrylthioacetamide was then oxidized with hydrogen peroxide in the presence of acetic acid to produce modafinil. Example 1a of '290 describes an alternate synthetic method of producing modafinil on an industrial scale. According to this process, benzhydrol is reacted with thiourea to form a compound which is subsequently hydrolyzed to benzhydrylthioacetic acid. The acid is then oxidized with hydrogen peroxide in a mixture of chloroacetic acid and water. The resulting modafinil-sulfoxide intermediate is treated with dimethyl sulfate to methylate the carboxylic acid group, and the resulting ester is derivatized with ammonia to produce modafinil.

In the processes disclosed by Lafon in ('290), the sulfide atoms in the modafinil intermediate compounds benzhydrylthioacetamide and benzhydrylthioacetic acid are oxidized with hydrogen peroxide. This oxidation process is carried out in acetic acid to solubilize the modafinil intermediate compounds. Singer et al., however, disclosed in U.S. Pat. No. 6,849,120 that this process tends to overoxidize the sulfide atom to produce the sulfone impurity benzhydrylsulfonylacetamide:

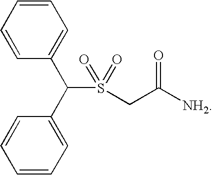

The overoxidation of the sulfide atom to sulfone occurs relatively consistently and strongly, particularly near the end of the reaction. Moreover, the sulfone impurity benzhydrylsulfonylacetamide is not easily removed from the pure modafinil product, resulting in elevated levels of impurities and reduced overall yield.

In addition to overoxidation, the use of substantial amounts of glacial acetic acid during the oxidation process is undesirable due to associated material handling issues. For example, the waste handling of glacial acetic acid following oxidation requires relatively large amounts of base and relatively long periods of time to neutralize safely.

In U.S. Pat. No. 6,849,120, Singer et al. disclosed a process for the preparation of modafinil in ≧99.5% purity after a single recrystallization. The process comprised oxidizing 2-[(diphenylmethyl)thio]acetamide (i.e., benzhydrylthioacetamide) with hydrogen peroxide in the presence of a mineral acid (preferably sulfuric acid) and either a linear, branched or cyclic alcohol, or a phase transfer catalyst. An inert liquid organic medium (such as methanol, ethanol, and ethylene glycol) was also used as a diluent for the oxidation reaction. Singer et al. described contacting 2-[(diphenylmethyl)thio]acetamide with from about 1.5 to about 4 molar equivalents of hydrogen peroxide. The mineral acid was described as being present in only a catalytic amount, preferably from about 0.002 to about 0.2 molar equivalents with respect to the acetamide. The alcohol or phase transfer catalyst was described as being used in an amount of from about 2 to about 4 equivalents with respect to the acetamide. While this procedure is generally effective in producing modafinil, there are a number of associated yield and material handling issues in using the relatively toxic sulfuric acid as a preferred mineral acid. The volume of water and sodium bisulfite necessary to neutralize the highly acidic mineral acid may tend to negatively effect productivity (e.g., the water and bisulfite dilute the reaction mixture, reducing yield, and the additional volume of reagents take up reactor space, effecting the economics of manufacture). Furthermore, the use of phase transfer catalysts in the reaction mixture may result in the presence of difficult-to-remove metal impurities in the final product.

A need persists for methods of producing modafinil and analogs thereof wherein the presence of impurities are minimized. It would be particularly desirable to provide an improved process for the synthesis of modafinil and analogs thereof that substantially minimizes the overoxidation of the sulfide atom to sulfone and provides a highly pure modafinil product prior to recrystallization. Additionally, it would be desirable to provide a process for producing modafinil and analogs thereof which minimizes the use of acetic acid and other harmful or toxic reagents in the oxidation reaction.

### SUMMARY OF THE INVENTION

Among the various aspects of the present invention is the provision of a process for the preparation of modafinil and analogs thereof. More specifically, the process involves oxidizing a modafinil intermediate compound in a mixture of reagents. The process minimizes the overoxidation of the sulfide atom to sulfone, thus minimizing impurities and improving overall yield. The process also utilizes relatively small amounts of an organic acid, as compared to known methods.

Briefly, therefore, the present invention is directed to a process for the preparation of modafinil or analogs thereof, the process comprising:

oxidizing a modafinil intermediate compound in a reaction mixture comprising an alcohol, an organic acid, and an oxidizing agent; and

recovering modafinil or analogs thereof from the reaction mixture; wherein

the ratio of alcohol to organic acid in the reaction mixture is from about 1:1 to about 80:1 (by volume);

the modafinil intermediate compound corresponds to Formula (1):

A-S—Y (1);

the recovered modafinil or analog thereof corresponds to Formula (10):

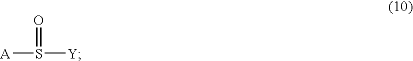

A is substituted alkyl, substituted aryl, substituted heteroaryl, or a substituted or unsubstituted tricyclic ring; and

Y is hydrocarbyl or substituted hydrocarbyl.

Other objects and features will be in part apparent and in part pointed out hereinafter.

### DETAILED DESCRIPTION OF THE INVENTION

The present invention is directed to a process for the preparation of modafinil and analogs thereof. The process includes the oxidation of a modafinil intermediate compound in a reaction mixture including an alcohol, an organic acid, and an oxidizing agent, wherein the alcohol and the organic acid are present in the reaction mixture at a ratio of from about 1:1 to about 80:1 (by volume). It has been found that by oxidizing a modafinil intermediate compound in a reaction mixture including an alcohol and an organic acid at these ratios, the overoxidation of the sulfide atom to sulfone is minimized and modafinil or analogs thereof may be recovered from the reaction mixture having high purity prior to recrystallization.

Modafinil and analogs thereof which can be prepared according to the process of the present invention generally correspond to Formula (10):

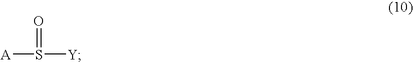

wherein A is substituted alkyl, substituted aryl, substituted heteroaryl, or a substituted or unsubstituted tricyclic ring; and Y is hydrocarbyl or substituted hydrocarbyl.

The process for producing modafinil and analogs thereof described above comprises oxidizing a modafinil intermediate compound corresponding to Formula (1):

A-S—Y (1)

in a reaction mixture comprising an alcohol, an organic acid, and an oxidizing agent, wherein A and Y are defined as above.

The oxidation of the modafinil intermediate compound corresponding to Formula (1) to produce modafinil and analogs thereof corresponding to Formula (10) generally proceeds according to Reaction Scheme 1:

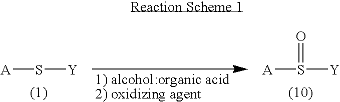

The designation “alcohol:organic acid” in the various reaction schemes herein refers to a ratio of alcohol to organic acid in the reaction mixture according to those described herein (e.g., from about 1:1 to about 80:1 (by volume).

Various modafinil intermediate compounds can be oxidized according to the process of the present invention to produce modafinil and analogs thereof according to Reaction Scheme 1.

In one embodiment, A is an alkylene substituted with two phenyl groups and Y is —(CH₂)—C(═O)—Y₁, wherein Y₁ is hydrocarbyl, hydroxy, halo, alkoxy, or amino. According to this embodiment, the modafinil intermediate compound corresponds to Formula (2):

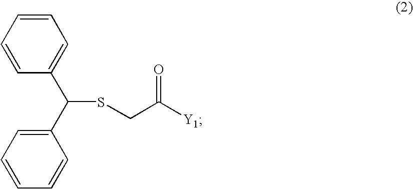

and the recovered modafinil or analog thereof corresponds to Formula (20):

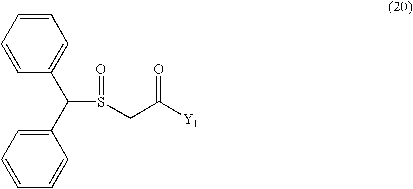

wherein Y₁ is as defined above.

In one preferred embodiment, Y₁ is —NH₂. According to this embodiment, the modafinil intermediate compound is benzhydrylthioacetamide (2A):

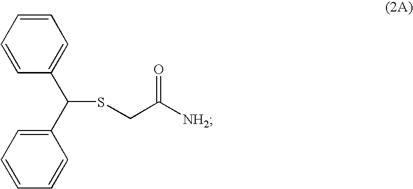

and the recovered modafinil or analog thereof is modafinil (200):

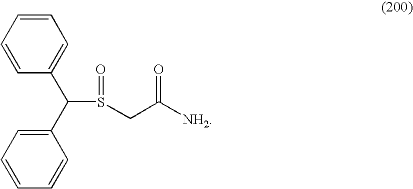

The modafinil intermediate compound benzhydrylthioacetamide (2A) can be produced according to various processes, such as those described in U.S. Pat. Nos. 4,177,290, 4,098,824, and 4,066,686 to Lafon; U.S. Pat. No. 6,875,893 to Largeu et al.; U.S. Pat. No. 6,649,796 to Naddaka et al., and WO2004/075841 and WO2005/042479 to Liang, each of which is hereby incorporated by reference herein. To produce benzhydrylthioacetamide (2A), several of these references describe first forming a benzhydrylthiouronium salt (A) from the reaction of benzhydrol, thiourea, and an acid (typically a hydrogen halide such as HCl or HBr), as illustrated in Reaction Scheme 2:

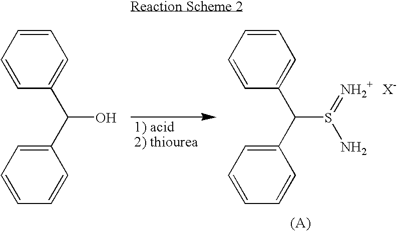

wherein X⁻ is the counterion from the corresponding acid.

The above-cited references then describe various synthesis routes using the benzhydrylthiouronium salt (A) to ultimately arrive at the modafinil intermediate compound benzhydrylthioacetamide (2A), which can then be oxidized according to the process of the present invention. Reaction Schemes 3(a)-(d) illustrate the various synthesis routes by which benzhydrylthioacetamide (2A) may be synthesized from benzhydrylthiouronium salt (A). Reaction Scheme 4 illustrates the oxidation of benzhydrylthioacetamide (2A) to produce modafinil (200) according to the process of the present invention.

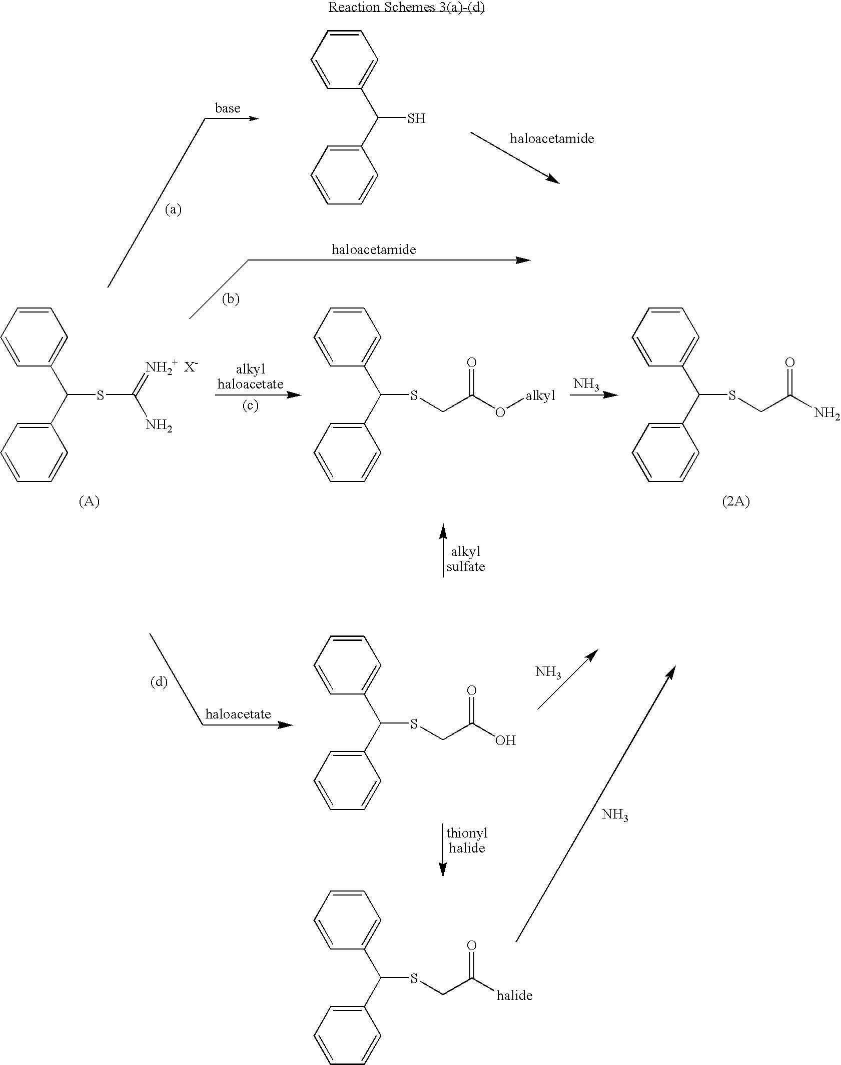

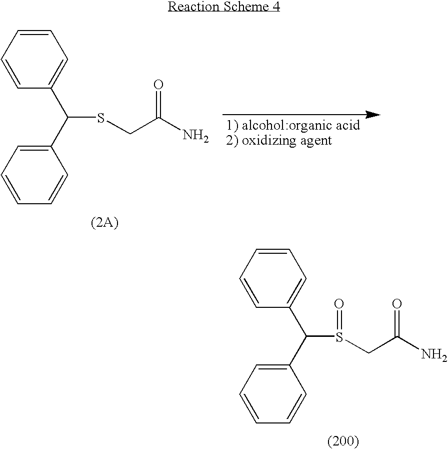

As noted above, when the modafinil intermediate compound corresponds to Formula (2), Y₁ may also be hydrocarbyl, hydroxy, halo, or alkoxy. According to this embodiment, the modafinil intermediate compounds may correspond to Formulae (2B), (2C), or (2D), which illustrate benzhydrylthioacetic acid, benzhydrylthioacetyl halide, or alkyl benzhydrylthioacetate, respectively.

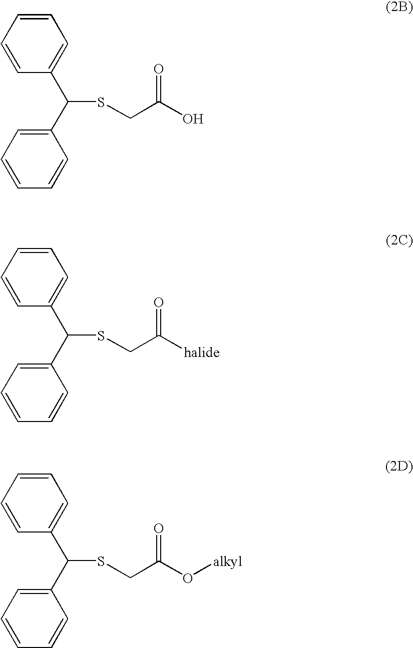

Reaction Schemes 5-8 illustrate processes for producing modafinil (200) wherein modafinil intermediate compounds (2B), (2C), (2D) above are oxidized according to the process of the present invention to produce various modafinil-sulfoxide intermediates. In Reaction Schemes 5-8, the general processes for producing the various intermediate compounds are the same or similar to those shown in Reaction Schemes 3(a)-(d), the only difference being that the oxidation step according to the present invention is performed at different steps (e.g., earlier) in the synthesis process. After oxidation, the modafinil-sulfoxide intermediates may then be then further derivatized to produce modafinil (200).

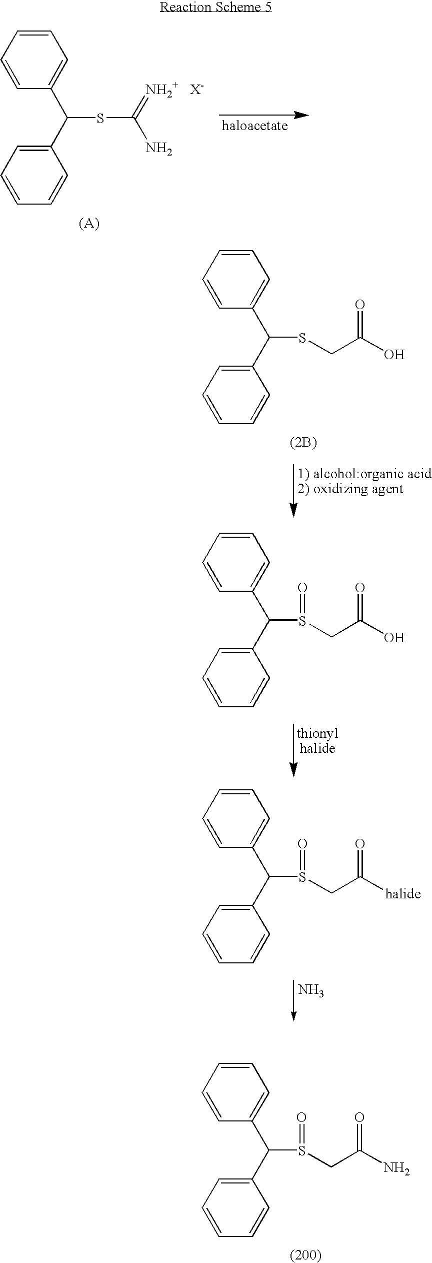

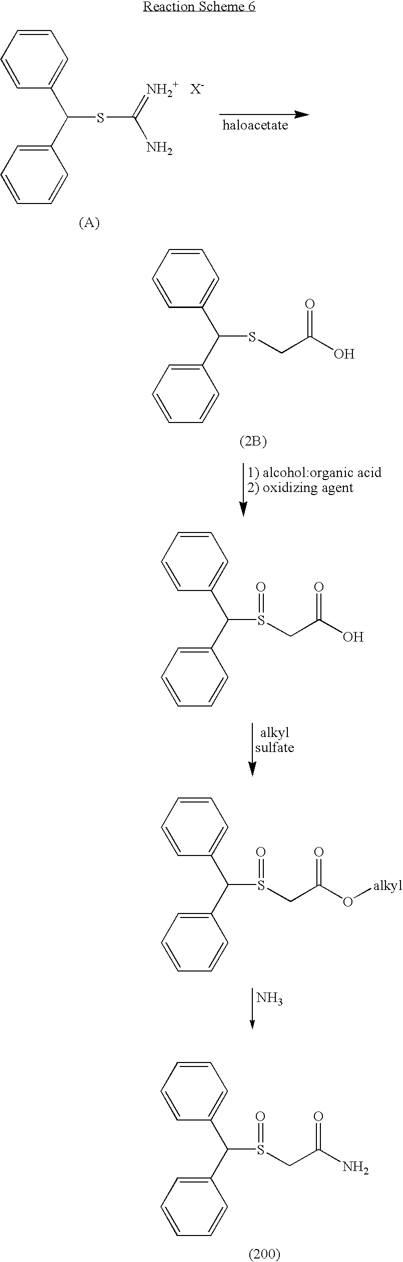

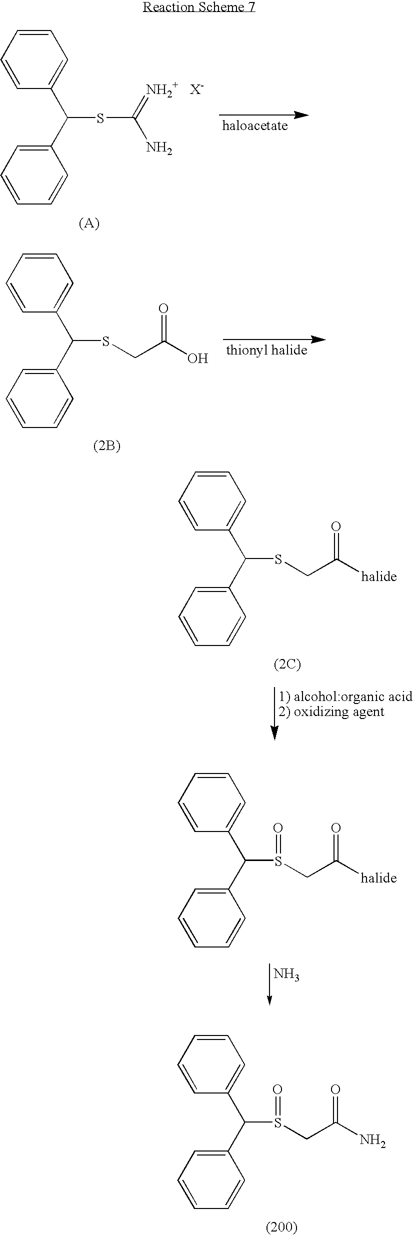

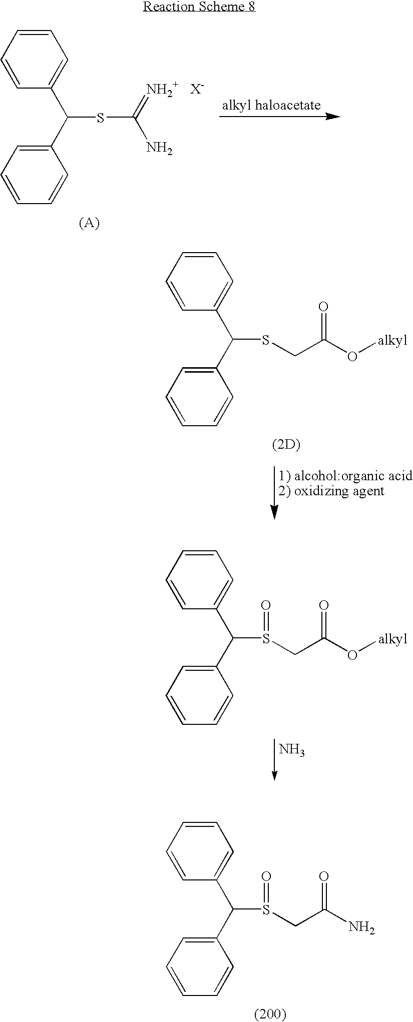

In other various embodiments, modafinil analogs may also be produced according to the process of the present invention by the oxidation of a modafinil intermediate compound in a reaction mixture comprising an alcohol, an organic acid, and an oxidizing agent, wherein the ratio of alcohol to organic acid in the reaction mixture is from about 1:1 to about 80:1 (by volume).

In one embodiment, the modafinil intermediate compound corresponds to Formula (3):

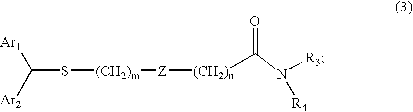

the recovered modafinil or analog thereof corresponds to Formula (30):

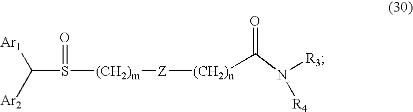

Ar₁ and Ar₂ are each independently selected from C₆-C₁₀ aryl or heteroaryl; wherein each of Ar₁ or Ar₂ may be independently optionally substituted with 1-3 substituents independently selected from:

a) H, C₆-C₁₀ aryl, heteroaryl, F, Cl, Br, I, —CN, —CF₃, —NO₂, —OH, —OR₇, —O(CH₂)ₚNR₉R₁₀, —OC(═O)R₇, —OC(═O)NR₉R₁₀, —O(CH₂)ₚOR₈, —CH₂OR₈, —NR₉R₁₀, —NR₈S(═O)₂R₇, —NR₈C(═O)R₇, or —NR₈C(═S)R₇;

b) —CH₂OR₁₁;

c) —NR₈C(═O)NR₉R₁₀, —NR₈C(═S)NR₉R₁₀, —CO₂R₁₂, —C(═O)R₁₃, —C(═O)NR₉R₁₀, —C(═S)NR₉R₁₀, —CH═NOR₁₂, —CH═NR₇, —(CH₂)ₚNR₉R₁₀, —(CH₂)ₚNHR₁₁, —CH═NNR₁₂R_{12A}, —C(═NR₈)NR_{8A}R_{8B}, —NR₈C(═NH)R_{8A}, —NR₈C(═NH)NR_{8A}R_{8B},

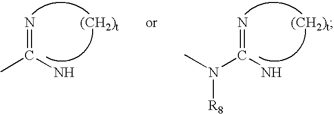

d) —S(O)_{y}R₁₇—(CH₂)ₚS(O)_{y}R₇, —CH₂S(O)_{y}R₇; and

e) C₁-C₈ alkyl, C₂-C₈ alkenyl, or C₂-C₈ alkynyl, where:

1) each alkyl, alkenyl, or alkynyl group is unsubstituted; or

2) each alkyl, alkenyl or alkynyl group is independently substituted with 1 to 3 groups independently selected from C₆-C₁₀ aryl, heteroaryl, F, Cl, Br, I, CF₃, —CN, —NO₂, —OH, —OR₇, —CH₂OR₈, —NR₉R₁₀, —O—(CH₂)ₚ—OH, —S—(CH₂)ₚ—OH, —X₁(CH₂)ₚOR₇, —X₁(CH₂)ₚNR₉R₁₀, —X₁(CH₂)ₚC(═O)NR₉R₁₀, —X₁(CH₂)ₚC(═S)NR₉R₁₀, —X₁(CH₂)ₚOC(═O)NR₉R₁₀, —X₁(CH₂)ₚCO₂R₈, —X₁(CH₂)ₚS(O)_{y}R₇, —X₁S(CH₂)ₚNR₈C(═O)NR₉R₁₀, —C(═O)R₁₃, —CO₂R₁₂, —OC(═O)R₇, —C(═O)NR₉R₁₀, —OC(═O)NR₁₂R_{12A}, O-tetrahydropyranyl, —C(═S)NR₉R₁₀, —CHNNR₁₂R_{12A}, —CHNOR₁₂, —CHNR₇, —CH═NNHCH(N═NH)NH₂, —NR₈CO₂R₇, —NR₈C(═O)NR₉R₁₀, —NR₈C(═S)NR₉R₁₀, —NHC(═NH)NH₂, —NR₈C(═O)R₇, —NR₈C(═S)R₇, —NR₈S(═O)₂R₇, —S(O)_{y}R₇, —S(═O)₂NR₁₂R_{12A}, —P(═O)(OR₈)₂, —OR₁₁, and a C₅-C₇ monosaccharide where each hydroxyl group of the monosaccharide is independently either unsubstituted or is replaced by H, C₁-C₄ alkyl, C₁-C₄ alkoxy, or —O—C(═O)R₇;

X₁ is —O—, —S—, or —N(R₈)—;

Z is selected from C₁-C₄ alkylene, —C(R₁)(R₂)—, C₆-C₁₀ arylene, heteroarylene, C₃-C₈ cycloalkylene, heterocyclylene, —O—, —N(R₈)—, —S(O)_{y}, —CR_{9A}═CR_{8B}—, —CH═CH—CH(R₈ₐ)—, —CH(R₈)—CH═CH—, or —C≡C—;

R₁, R₂, R₃ and R₄ are each independently selected from H, C₁-C₆ alkyl, —OH, and —CH(R₈)—CONR_{8A}R_{8B}; or R₃ and R₄, together with the nitrogen to which they are attached, form a 3-7 member heterocyclyl ring;

R₆ is H, C₁-C₄ alkyl, or the side chain of an α-amino acid;

R₇ is C₁-C₆ alkyl, C₆-C₁₀ aryl, or heteroaryl;

R₈, R_{8A} and R_{8B} are each independently H, C₁-C₄ alkyl, or C₆-C₁₀ aryl;

R₉ and R₁₀ are each independently selected from H, C₁-C₄ alkyl, and C₆-C₁₀ aryl; or R₉ and R₁₀ together with the nitrogen to which they are attached, form a 3-7 member heterocyclyl ring;

R₁₁ is the residue of an amino acid after the hydroxyl group of the carboxyl group is removed;

R₁₂ and R_{12A} are each independently selected from H, C₁-C₆ alkyl, cycloalkyl, C₆-C₁₀ aryl, and heteroaryl; or R₁₂ and R_{12A}, together with the nitrogen to which they are attached, form a 5-7 member heterocyclyl ring;

R₁₃ is H, C₁-C₆ alkyl, cycloalkyl, C₆-C₁₀ aryl, heteroaryl, —C(═O)R₇, —C(═O)NR₉R₁₀, or —C(═S)NR₉R₁₀;

m is 0, 1, 2 or 3;

n is 0, 1, 2 or 3;

p is 1, 2, 3 or 4;

t is 2, 3 or 4; and

y is 0, 1 or 2.

In another embodiment, the modafinil intermediate compound corresponds to Formula (4):

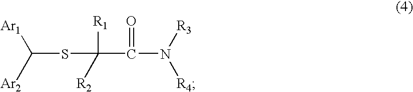

the recovered modafinil or analog thereof corresponds to Formula (40):

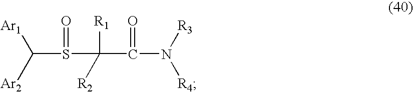

Ar₁ and Ar₂ are each independently selected from thiophene, isothiazole, phenyl, pyridyl, oxazole, isoxazole, thiazole, imidazole, and other five or six membered heterocycles comprising 1-3 atoms of —N—, —O—, or —S—;

R₁, R₂, R₃ and R₄ are each independently selected from H, lower alkyl, —OH, —CH(R₆)—CONR_{6A}R_{6B}, or any of R₁, R₂, R₃ and R₄ can be taken together to form a 3-7 member carbocyclic or heterocyclic ring; and

each of Ar₁ or Ar₂ may be independently optionally substituted with one or more substituents independently selected from:

a) H, aryl, heterocyclyl, F, Cl, Br, I, —CN, —CF₃, —NO₂, —OH, —OR₇, —O(CH₂)ₚNR₉R₁₀, —OC(═O)R₇, —OC(═O)NR₉R₁₀, —O(CH₂)ₚOR₈, —CH₂OR₈, —NR₉R₁₀, —NR₈S(═O)₂R₇, —NR₈C(═O)R₇, or —NR₈C(═S)R₇;

b) —CH₂OR₁₁, where R₁₁ is the residue of an amino acid after the hydroxyl group of the carboxyl group is removed;

c) —NR₈C(═O)NR₉R₁₀, —NR₈C(═S)NR₉R10, —CO₂R₁₂, —C(═O)R₁₂, —C(═O)NR₉R₁₀, —C(═S)NR₉R₁₀, —CH═NOR₁₂, —CH═NR₇, —(CH₂)ₚNR₉R₁₀, —(CH₂)ₚNHR₁₁, or —CH═NNR₁₂R_{12A}, where R₁₂ and R_{12A} are each independently selected from H, alkyl of 1 to 4 carbons, —OH, alkoxy of 1 to 4 carbons, —OC(═O)R₇, —OC(═O)NR₉R₁₀, —OC(═S)NR₉R₁₀, —O(CH₂)ₚNR₉R₁₀, —O(CH₂)ₚOR₈, substituted or unsubstituted arylalkyl having from 6 to 10 carbons, and substituted or unsubstituted heterocyclylalkyl;

d) —S(O)_{y}R₁₂, —(CH₂)ₚS(O)_{y}R₇, —CH₂S(O)_{y}R₁₁ where y is 0, 1 or 2; and

e) alkyl of 1 to 8 carbons, alkenyl of 2 to 8 carbons, or alkynyl of 2 to 8 carbons, where:

1) each alkyl, alkenyl, or alkynyl group is unsubstituted; or

2) each alkyl, alkenyl or alkynyl group is substituted with 1 to 3 groups selected from aryl of 6 to 10 carbons, heterocyclyl, arylalkoxy, heterocycloalkoxy, hydroxylalkoxy, alkyloxy-alkoxy, hydroxyalkylthio, alkoxy-alkylthio, F, Cl, Br, I, —CN, —NO₂, —OH, —OR₇, —X₂(CH₂)ₚNR₉R₁₀, —X₂(CH₂)ₚC(═O)NR₉R₁₀, —X₂(CH₂)ₚC(═S)NR₉R₁₀, —X₂(CH₂)ₚOC(═O)NR₉R₁₀, —X₂(CH₂)ₚCO₂R₇, —X₂(CH₂)ₚS(O)_{y}R₇, —X₂(CH₂)ₚNR₈C(═O)NR₉R₁₀, —OC(═O)R₇, —OC(═O)NHR₁₂, O-tetrahydropyranyl, —NR₉R₁₀, —NR₈CO₂R₇, —NR₈C(═O)NR₉R₁₀, —NR₈C(═S)NR₉R₁₀, —NHC(═NH)NH₂, —NR₈C(═O)R₇, —NR₈C(═S)R₇, —NR₈S(═O)₂R₇, —S(O)_{y}R₇, —CO₂R₁₂, —C(═O)NR₉R₁₀, —C(═S)NR₉R₁₀, —C(═O)R₁₂, —CH₂OR₈, —CH═NNR₁₂R_{12A}, —CH═NOR₁₂, —CH═NR₇, —CH═NNHCH(N═NH)NH₂, —S(═O)₂NR₁₂R_{12A}, —P(═O)(OR₈)₂, —OR₁₁, and a monosaccharide of 5 to 7 carbons where each hydroxyl group of the monosaccharide is independently either unsubstituted or is replaced by H, alkyl of 1 to 4 carbons, alkylcarbonyloxy of 2 to 5 carbons, or alkoxy of 1 to 4 carbons, where X₂ is O, S, or NR₈; where

R₇ is substituted or unsubstituted alkyl, substituted or unsubstituted aryl, or substituted or unsubstituted heterocyclyl;

R₈ is H or alkyl having from 1 to 4 carbons;

p is 1, 2, 3 or 4; and where either

1) R₉ and R₁₀ are each independently H, unsubstituted alkyl of 1 to 4 carbons, or substituted alkyl; or

2) R₉ and R₁₀ together form a linking group of the formula —(CH₂)₂—X₁—(CH₂)₂—, wherein X₁ is selected from —O—, —S—, and —CH₂—.

In another embodiment, the modafinil intermediate compound corresponds to Formula (5):

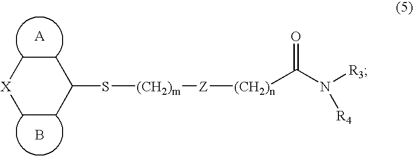

the recovered modafinil or analog thereof corresponds to Formula (50):

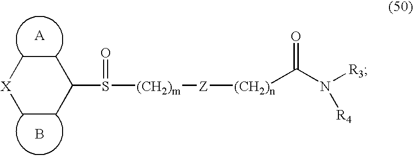

X is a bond, —CH₂CH₂—, —O—, S(O)_{y}—, —N(R₈)—, —CHN(R₈)—, —CH═CH—, —CH₂—CH═CH—, C(═O), —C(R₈)═N—, —N═C(R₈), —C(═O)—N(R₈)—, or —NR₈—C(═O)—;

Rings A and B, together with the carbon atoms to which they are attached, are each independently selected from:

(a) a 6-membered aromatic carbocyclic ring in which from 1 to 3 carbon atoms may be replaced by hetero atoms selected from oxygen, nitrogen and sulfur; and

b) a 5-membered aromatic carbocyclic ring in which either:

i) one carbon atom is replaced with an oxygen, nitrogen, or sulfur atom;

ii) two carbon atoms are replaced with a sulfur and a nitrogen atom, an oxygen and a nitrogen atom, or two nitrogen atoms; or

iii) three carbon atoms are replaced with three nitrogen atoms, one oxygen and two nitrogen atoms, or one sulfur and two nitrogen atoms;

wherein Ring A and Ring B may each be independently substituted with 1-3 substituents selected from:

a) H, C₆-C₁₀ aryl, heteroaryl, F, Cl, Br, I, —CN, —CF₃, —NO₂, —OH, —OR₇, —O(CH₂)ₚ NR₉R₁₀—OC(═O)R₇, —OC(═O)NR₉R₁₀, —O(CH₂)ₚOR₈, —CH₂OR₈, —NR₉R₁₀, —NR₈S(═O)₂R₇, —NR₈C(═O)R₇, or —NR₈C(═S)R₇;

b) —CH₂OR₁₁;

c) —NR₈C(═O)NR₉R₁₀, —NR₈C(═S)NR₉R₁₀, —CO₂R₁₂, —C(═O)R₁₃, —C(═O)NR₉R₁₀, —C(═S)NR₉R₁₀, —CH═NOR₁₂, —CH═NR₇, —(CH₂)ₚNR₉R₁₀, —(CH₂)ₚNHR₁₁, —CH═NNR₁₂R_{12A}, —C(═NR₈)NR_{8A}R_{8B}—NR₈C(═NH)R_{8A}, —NR₈C(═NH)NR_{8A}R_{8B},

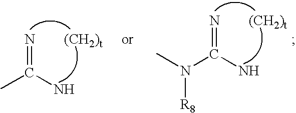

d) —S(O)_{y}R₇, —(CH₂)ₚS(O)_{y}R₇, —CH₂S(O)_{y}R₇; and

e) C₁-C₈ alkyl, C₂-C₈ alkenyl, or C₂-C₈ alkynyl, where:

1) each alkyl, alkenyl, or alkynyl group is unsubstituted; or

2) each alkyl, alkenyl or alkynyl group is independently substituted with 1 to 3 groups independently selected from C₆-C₁₀ aryl, heteroaryl, F, Cl, Br, I, CF₃, —CN, —NO₂, —OH, —OR₇, —CH₂OR₈, —NR₉R₁₀, —O—(CH₂)ₚ—OH, —S—(CH₂)ₚ—OH, —X₁(CH₂)ₚOR₇, X₁(CH₂)ₚNR₉R₁₀, —X₁(CH₂)ₚC(═O)NR₉R₁₀, —X₁ (CH₂)ₚC(═S)NR₉R₁₀, —X₁ (CH₂)ₚOC(═O)NR₉R₁₀, —X₁(CH₂)ₚCO₂R₈, —X₁(CH₂)ₚS(O)_{y}R₇, —X₁(CH₂)ₚNR₈C(═O)NR₉R₁₀, —C(═O)R₁₃, —CO₂R₁₂, —OC(═O)R₇, —C(═O)NR₉R₁₀, —OC(═O)NR₁₂R_{12A}, O-tetrahydropyranyl, —C(═S)NR₉R₁₀, —CH═NNR₁₂R_{12A}, —CH═NOR₁₂, —CH═N₇, —CH═NNHCH(N═NH)NH₂, —NR₈CO₂R₇, —NR₈C(═O)NR₉R₁₀, —NR₈C(═S)NR₉R₁₀, —NHC(═NH)NH₂, —NR₈C(═O)R₇, —NR₈C(═S)R₇, —NR₈S(═O)₂R₇, —S(O)_{y}R₇, —S(═O)₂NR₁₂R_{12A}, —P(═O)(OR₈)₂, —OR₁₁, and a C₅-C₇ monosaccharide where each hydroxyl group of the monosaccharide is independently either unsubstituted or is replaced by H, C₁-C₄ alkyl, C₁-C₄ alkoxy, or —O—C(═O)R₇;

R₃ and R₄ are each independently selected from H, C₁-C₆ alkyl, —OH, —CH(R₆)—CONR_{8A}R_{8B}, or R₃ and R₄, together with the nitrogen to which they are attached, form a 3-7 member heterocyclic ring;

R₆ is H, C₁-C₄ alkyl or the side chain of an α-amino acid;

R₇ is C₁-C₆ alkyl, C₆-C₁₀ aryl, or heteroaryl;

R₈, R_{8A} and R_{8B} are each independently H, C₁-C₄ alkyl, or C₆-C₁₀ aryl;

R₉ and R₁₀ are each independently selected from H, C₁-C₄ alkyl, and C₆-C₁₀ aryl; or R₉ and R₁₀ together with the nitrogen to which they are attached, form a 3-7 member heterocyclic ring;

R₁₁ is the residue of an amino acid after the hydroxyl group of the carboxyl group is removed;

R₁₂ and R_{12A} are each independently selected from H, C₁-C₆ alkyl, cycloalkyl, C₆-C₁₀ aryl, and heteroaryl; or R₁₂ and R_{12A}, together with the nitrogen to which they are attached, form a 5-7 member heterocyclic ring;

R₁₃ is H, C₁-C₆ alkyl, cycloalkyl, C₆-C₁₀ aryl, heteroaryl, —C(═O)R₇, —C(═O)NR₉R₁₀, or —C(═S)NR₉R₁₁;

X₁ is —O—, —S—, or —N(R₈)—;

Z is selected from C₁-C₄alkylene, C₆-C₁₀arylene, heteroarylene, C₃-C₈ cycloalkylene, heterocyclylene, —O—, —N(R₈)—, —S(O)_{y}, —CR_{8A}═CR_{8B}—, —CH═CH—CH(R₈)—, —CH(R₈)—CH═CH—, or —C≡C—;

m is 0, 1, 2 or 3;

n is 0, 1, 2 or 3;

p is 1, 2, 3 or 4;

q is 0, 1 or 2;

t is 2, 3 or 4; and

y is 0, 1 or 2.

In yet another embodiment, the modafinil intermediate compound corresponds to Formula (6):

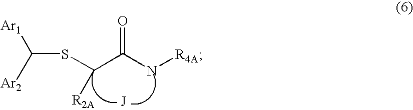

the recovered modafinil or analog thereof corresponds to Formula (60):

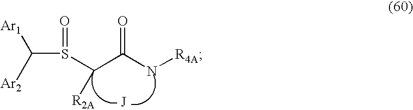

Ar₁ and Ar₂ are each independently selected from C₆-C₁₀ aryl or heteroaryl; wherein each of Ar₁ or Ar₂ may be independently optionally substituted with 1-3 substituents independently selected from:

a) H, C₆-C₁₀ aryl, heteroaryl, F, Cl, Br, I, —CN, —CF₃, —NO₂, —OH, —OR₇, —O(CH₂)ₚNR₉R₁₀, —OC(═O)R₇, —OC(═O)NR₉R₁₀, —O(CH₂)ₚOR₈, —CH₂OR₈, —NR₉R₁₀, —NR₈S(═O)₂R₇, —NR₈C(═O)R₇, or —NR₈C(═S)R₇;

b) —CH₂OR₁₁;

c) —NR₈C(═O)NR₉R₁₀, —NR₈C(═S)NR₉R₁₀, —CO₂R₁₂, —C(═O)R₁₃, —C(═O)NR₉R₁₀, —C(═S)NR₉R₁₀, —CH═NOR₁₂, —CH═NR₇, —(CH₂)ₚNR₉R₁₀, —(CH₂)ₚNHR₁₁, —CH═NNR₁₂R_{12A}, —C(═NR₈)NR_{8A}R_{8B}, —NR₈C(═NH)R_{8A}, —NR₈C(═NH)NR_{8A}R_{8B},

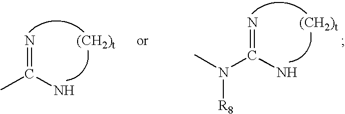

d) —S(O)_{y}R₇, —(CH₂)ₚS(O)_{y}R₇, —CH₂S(O)_{y}R₇; and

e) C₁-C₈ alkyl, C₂-C₈ alkenyl, or C₂-C₈ alkynyl, where:

1) each alkyl, alkenyl, or alkynyl group is unsubstituted; or

2) each alkyl, alkenyl or alkynyl group is independently substituted with 1 to 3 groups independently selected from C₆-C₁₀ aryl, heteroaryl, F, Cl, Br, I, CF₃, —CN, —NO₂, —OH, —OR₇, —CH₂OR₈, —NR₉R₁₀, —O—(CH₂)ₚ—OH, —S—(CH₂)ₚ—OH, —X₁(CH₂)ₚOR₇, —X₁(CH₂)ₚNR₉R₁₀, —X₁(CH₂)ₚC(═O)NR₉R₁₀, —X₁(CH₂)ₚC(═S)NR₉R₁₀, —X₁(CH₂)ₚOC(═O)NR₉R₁₀, —X₁(CH₂)ₚCO₂R₈, —X₁(CH₂)ₚS(O)_{y}R₇, —X₁S(CH₂)ₚNR₈C(═O)NR₉R₁₀, —C(═O)R₁₃, —CO₂R₁₂, —OC(═O)R₇, —C(═O)NR₉R₁₀, —OC(═O)NR₁₂R_{12A}, O-tetrahydropyranyl, —C(═S)NR₉R₁₀, —CHNNR₁₂R_{12A}, —CHNOR₁₂, —CHNR₇, —CH═NNHCH(N═NH)NH₂, —NR₈CO₂R₇, —NR₈C(═O)NR₉R₁₀, —NR₈C(═S)NR₉R₁₀, —NHC(—NH)NH₂, —NR₈C(═O)R₇, —NR₈C(═S)R₇, —NR₈S(═O)₂R₇, —S(O)_{y}R₇, —S(═O)₂NR₁₂R_{12A}, —P(═O)(OR₈)₂, —OR₁₁, and a C₅-C₇ monosaccharide where each hydroxyl group of the monosaccharide is independently either unsubstituted or is replaced by H, C₁-C₄ alkyl, C₁-C₄ alkoxy, or —O—C(═O)R₇;

X₁ is —O—, —S—, or —N(R₈)—;

J is C₂-C₄ alkylene or Q-CO—;

Q is C₁-C₃ alkylene;

R_{2A} is H, C₁-C₆ alkyl, aryl or heteroaryl;

R_{4A} is H, C₁-C₆ alkyl, aryl or heteroaryl;

R₇ is C₁-C₆ alkyl, C₆-C₁₀ aryl, or heteroaryl;

R⁸, R_{8A} and R_{8B} are each independently H, C₁-C₄ alkyl, or C₆-C₁₀ aryl;

R₉ and R₁₀ are each independently selected from H, C₁-C₄ alkyl, and C₆-C₁₀ aryl; or R₉ and R₁₀ together with the nitrogen to which they are attached, form a 3-7 member heterocyclic ring;

R₁₁ is the residue of an amino acid after the hydroxyl group of the carboxyl group is removed;

R₁₂ and R_{12A} are each independently selected from H, C₁-C₆ alkyl, cycloalkyl, C₆-C₁₀ aryl, and heteroaryl; or R₁₂ and R_{12A}, together with the nitrogen to which they are attached, form a 5-7 member heterocyclic ring;

R₁₃ is H, C₁-C₆alkyl, cycloalkyl, C₆-C₁₀aryl, heteroaryl, —C(═O)R₇, —C(═O)NR₉R₁₀, or —C(═S)NR₉R₁₀;

p is 1, 2, 3 or 4;

q is 0, 1 or 2;

t is 2, 3 or 4; and

y is 0, 1 or 2.

In yet another embodiment, the modafinil intermediate compound corresponds to Formula (7):

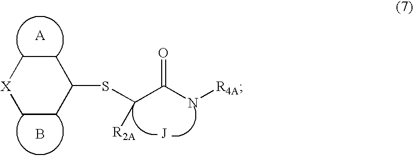

the recovered modafinil or analog thereof corresponds to Formula (70):

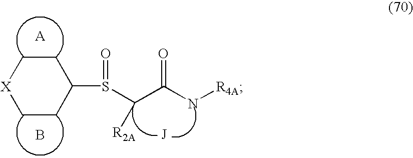

X is a bond, —CH₂CH₂—, —O—, S(O)_{y}—, —N(R₈)—, —CHN(R₈)—, —CH═CH—, —CH₂—CH═CH—, C(═O), —C(R₈)═N—, —N═C(R₈)—, —C(═O)N(R₈)—, or —NR₈—C(═O)—;

Rings A and B, together with the carbon atoms to which they are attached, are each independently selected from:

(a) a 6-membered aromatic carbocyclic ring in which from 1 to 3 carbon atoms may be replaced by hetero atoms selected from oxygen, nitrogen and sulfur; and

b) a 5-membered aromatic carbocyclic ring in which either:

i) one carbon atom is replaced with an oxygen, nitrogen, or sulfur atom;

ii) two carbon atoms are replaced with a sulfur and a nitrogen atom, an oxygen and a nitrogen atom, or two nitrogen atoms; or

iii) three carbon atoms are replaced with three nitrogen atoms, one oxygen and two nitrogen atoms, or one sulfur and two nitrogen atoms;

wherein Ring A and Ring B may each be independently substituted with 1-3 substituents selected from:

a) H, C₆-C₁₀ aryl, heteroaryl, F, Cl, Br, I, —CN, —CF₃, —NO₂, —OH, —OR₇, —O(CH₂)ₚNR₉R₁₀, —OC(═O)R₇, —OC(═O)NR₉R₁₀, —O(CH₂)ₚOR₈, —CH₂OR₈, —NR₉R₁₀, —NR₈S(═O)₂R₇, —NR₈C(═O)R₇, or —NR₈C(═S)R₇;

b) —CH₂OR₁₁;

c) —NR₈C(═O)NR₉R₁₀, —NR₈C(═S)NR₉R₁₀, —CO₂R₁₂, —C(═O)R₁₃, —C(═O)NR₉R₁₀, —C(═S)NR₉R₁₀, —CH═NOR₁₂, —CH═NR₇, —(CH₂)ₚNR₉R₁₀, —(CH₂)ₚNHR₁₁, —CH═NNR₁₂R_{12A}, —C(═NR₈)NR_{8A}R_{8B}—NR₈C(═NH)R_{8A}, —NR₈C(═NH)NR_{8A}R_{8B},

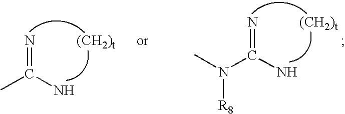

d) —S(O)_{y}R₇, —(CH₂)ₚS(O)_{y}R₇, —CH₂S(O)_{y}R₇; and

e) C₁-C₈ alkyl, C₂-C₈ alkenyl, or C₂-C₈ alkynyl, where:

1) each alkyl, alkenyl, or alkynyl group is unsubstituted; or

2) each alkyl, alkenyl or alkynyl group is independently substituted with 1 to 3 groups independently selected from C₆-C₁₀ aryl, heteroaryl, F, Cl, Br, I, CF₃, —CN, —NO₂, —OH, —OR₇, —CH₂OR₈, —NR₉R₁₀, —O—(CH₂)ₚ—OH, —S—(CH₂)ₚ—OH, —X₁(CH₂)ₚOR₇, X₁(CH₂)ₚNR₉R₁₀, —X₁ (CH₂)ₚC(═O)NR₉R₁₀, —X₁(CH₂)ₚC(═S)NR₉R₁₀, —X₁(CH₂)ₚOC(═O)NR₉R₁₀, —X₁(CH₂)ₚCO₂R₈, —X₁(CH₂)ₚS(O)_{y}R₇, —X₁(CH₂)ₚNR₈C(═O)NR₉R₁₀, —C(═O)R₁₃, —CO₂R₁₂, OC(═O)R₇, —C(═O)NR₉R₁₀, —OC(═O)NR₁₂R_{12A}, O-tetrahydropyranyl, —C(═S)NR₉R₁₀, —CH═NNR₁₂R_{12A}, —CH═NOR₁₂, —CH═N₇, —CH═NNHCH(N═NH)NH₂, —NR₈CO₂R₇, —NR₈C(═O)NR₉R₁₀, —NR₈C(═S)NR₉R₁₀, —NHC(═NH)NH₂, —NR₈C(═O)R₇, —NR₈C(═S)R₇, —NR₈S(═O)₂R₇, —S(O)_{y}R₇, —S(═O)₂NR₁₂R_{12A}, —P(═O)(OR₈)₂, —OR₁₁, and a C₅-C₇ monosaccharide where each hydroxyl group of the monosaccharide is independently either unsubstituted or is replaced by H, C₁-C₄ alkyl, C₁-C₄ alkoxy, or —O—C(═O)R₇;

J is C₂-C₄ alkylene or Q-CO—;

Q is C₁-C₃ alkylene;

R_{2A} is H, C₁-C₈ alkyl, aryl or heteroaryl;

R_{4A} is H, C₁-C₆ alkyl, aryl or heteroaryl;

R₇ is C₁-C₆ alkyl, C₆-C₁₀ aryl, or heteroaryl;

R₈, R_{8A} and R_{8B} are each independently H, C₁-C₄ alkyl, or C₆-C₁₀ aryl;

R₉ and R₁₀ are each independently selected from H, C₁-C₄ alkyl, and C₆-C₁₀ aryl; or R₉ and R₁₀ together with the nitrogen to which they are attached, form a 3-7 member heterocyclic ring;

R₁₁ is the residue of an amino acid after the hydroxyl group of the carboxyl group is removed;

R₁₂ and R_{12A} are each independently selected from H, C₁-C₆ alkyl, cycloalkyl, C₁-C₁₀ aryl, and heteroaryl; or R₁₂ and R_{12A}, together with the nitrogen to which they are attached, form a 5-7 member heterocyclic ring;

R₁₃ is H, C₁-C₈ alkyl, cycloalkyl, C₆-C₁₀ aryl, heteroaryl, —C(═O)R₇, —C(═O)NR₉R₁₀, or —C(═S)NR₉R₁₀;

X₁ is —O—, —S—, or —N(R₈)—;

p is 1, 2, 3 or 4;

q is 0, 1 or 2;

t is 2, 3 or 4; and

y is 0, 1 or 2.

In yet another embodiment, the modafinil intermediate compound corresponds to Formula (8):

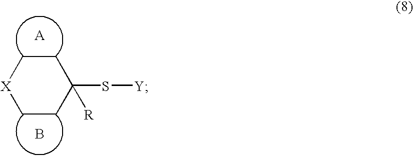

the recovered modafinil or analog thereof corresponds to Formula (80):

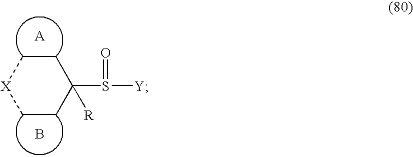

Rings A and B, together with the carbon atoms to which they are attached, are each independently selected from:

a) a 6-membered aromatic carbocyclic ring in which from 1 to 3 carbon atoms may be replaced by hetero atoms selected from oxygen, nitrogen and sulfur; and

b) a 5-membered aromatic carbocyclic ring in which either:

i) one carbon atom may be replaced with an oxygen, nitrogen, or sulfur atom;

ii) two carbon atoms may be replaced with a sulfur and a nitrogen atom, an oxygen and a nitrogen atom, or two nitrogen atoms; or

iii) three carbon atoms may be replaced with three nitrogen atoms, one oxygen and two nitrogen atoms, or one sulfur and two nitrogen atoms; wherein said rings are optionally substituted with one to three R²⁰ groups;

X is not present, is a bond, O, S(O)_{y}, NR¹⁰, C₂ alkylene, C₂₋₃ alkenylene, C(═O), C(R²¹)₂NR¹⁰, C(R²¹)═N, N═C(R²¹), C(═O)N(R²¹), or NR¹⁰C(═O); wherein said alkylene and alkenylene groups are optionally substituted with one to three R²⁰ groups;

R is H or C₁-C₆ alkyl;

Y is selected from:

a) C₁-C₆ alkylene-R¹;

b) C₁-C₆ alkylene-R²;

c) (C₁-C₄ alkylene)ₘ-Z-(C₁-C₄ alkylene)ₙ-R¹;

d) C₁-C₆ alkylene-O(CH₂)ₚOR²¹,

e) C₁-C₆ alkyl substituted with one or two OR²¹ groups; and

f) CH₂CR²¹═C(R²¹)₂;

wherein said alkyl and alkylene groups are optionally substituted with one to three R²⁰ groups;

Z is O, NR^{10A}, S(O)_{y}, CR²¹═CR²¹, C═C(R²¹)₂, C≡C, C₆-C₁₀ arylene, 5-10 membered heteroarylene, C₃-C₆ cycloalkylene, or 3-6 membered heterocycloalkylene; wherein said arylene, heteroarylene, cycloalkylene, and heterocycloalkylene groups are optionally substituted with one to three R²⁰ groups;

R¹ is selected from NR¹²R¹³, NR²¹C(═O)R¹⁴, C(═O)R¹⁵, CO₂R¹¹, OC(═O)R¹¹, C(═O)NR¹²R¹³, C(═O)NR²¹ OR¹⁴, C(═NR¹¹)NR¹²R¹³, NR²¹S(O)2R¹¹, S(O)₂NR¹²R¹³, NR²¹S(O)₂NR¹²R¹³, and PO(OR²¹)₂;

R² is a 5-6 membered heteroaryl, wherein said heteroaryl group is optionally substituted with one to three R²⁰ groups;

R¹⁰ and R^{10A} at each occurrence is independently selected from H, C₁-C₆ alkyl, C₆-C₁₀ aryl, C(═O)R¹⁵, and S(O)_{y}R¹⁴; wherein said alkyl and aryl groups are optionally substituted with one to three R²⁰ groups;

R¹⁴ at each occurrence is independently selected from C₁-C₆ alkyl, C₅-C₁₀ aryl, and arylalkyl; wherein said alkyl, aryl and arylalkyl groups are optionally substituted with one to three R²⁰ groups;

R¹⁵ at each occurrence is independently selected from C₁-C₆ alkyl, C₆-C₁₀ aryl, arylalkyl, and heteroaryl; wherein said alkyl, aryl, arylalkyl, and heteroaryl groups are optionally substituted with one to three R²⁰ groups;

R²⁰ at each occurrence is independently selected from F, Cl, Br, I, OR²¹, OR²⁵, NR²³R²⁴, NHOH, NO₂, CN, CF₃, C₁-C₆ alkyl, C₃-C₆ spirocycloalkyl, C₂-C₆ alkenyl, C₂-C₆ alkynyl, C₃-C₇ cycloalkyl, 3-7 membered heterocycloalkyl, phenyl, 5 or 6 membered heteroaryl, arylalkyl, ═O, C(═O)R²², CO₂R²¹, OC(═O)R²², C(═O)NR²³R²⁴, NR²¹C(═O)R²², NR²¹CO₂R²², OC(═O)NR²³R²⁴, NR²¹C(═O)R²², NR²¹C(═S)R²², and S(O)_{y}R²²;

R²¹ at each occurrence is independently selected from H and C₁-C₆ alkyl;

R²² at each occurrence is independently selected from C₁-C₆ alkyl and C₆-C₁₀ aryl;

R²³ and R²⁴ at each occurrence are each independently selected from H, C₁-C₆ alkyl, and C₆-C₁₀ aryl, or R²³ and R²⁴, together with the nitrogen to which they are attached, form a 3-7 membered heterocycloalkyl ring;

R²⁵ at each occurrence is independently the residue of an amino acid after the hydroxyl group of the carboxyl group is removed; and

y is 0, 1 or 2.

In another embodiment, the modafinil intermediate compound corresponds to Formula (9):

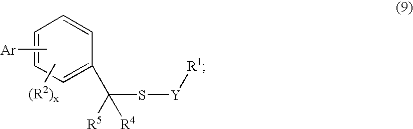

the recovered modafinil or analog thereof corresponds to Formula (90):

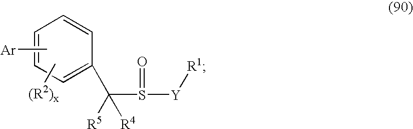

Ar is C₆-C₁₀ aryl substituted by 0-5 R₃; C₅-C₁₀ cycloalkenyl substituted by 0-5 R³; or 5 to 14 membered heteroaryl group substituted by 0-5 R³, wherein said heteroaryl group comprises one, two, or three heteroatoms selected from N, O, S or Se;

Y is C₁-C₆ alkylene substituted with 0-3 R^{20A};

R¹ is selected from H, C(═O)NR¹²R¹³, C(═N)NR¹²R¹³, OC(═O)NR¹²R¹³, NR²¹C(═O)NR¹²R¹³, NR²¹S(═O)₂NR¹²R¹³, —(C₆-C₁₀ aryl)-NR¹²R¹³ wherein said aryl is substituted with 0-3 R²⁰; NR²¹C(═O)R¹⁴, C(═O)R¹⁴, C(═O)OR¹¹, OC(═O)R¹¹, and NR²¹S(═O)₂R¹¹;

R² is selected from H, F, Cl, Br, I, OR¹⁶, OR²⁵, NR¹⁷R¹⁸, NHOH, NO₂, CN, CF₃, C₁-C₆ alkyl, C₂-C₆ alkenyl, C₂-C₆ alkynyl, C(═O)R¹⁶, C(═O)OR¹⁸, OC(═O)R¹⁶, C(═O)NR¹⁷R¹⁸, NR¹⁵C(═O)R¹⁶, NR¹⁵CO₂R¹⁶, OC(═O)NR¹⁷R¹⁸, NR¹⁵C(═S)R¹⁶, SR¹⁶; S(═O)R¹⁶; and S(═O)₂R¹⁶; alternatively, two R² groups may be combined to form a methylenedioxy group, an ethylenedioxy group, or a propylenedioxy group;

R³ is selected from H, F, Cl, Br, I, OR¹⁶, OCF₃, OR²⁵, NR¹⁷R¹⁸, NHOH, NO₂, CN, CF₃, CH₂OR¹⁶, C₁-C₆ alkyl, C₂-C₆ alkenyl, C₂-C₆ alkynyl, C₃-C₇ cycloalkyl, 3-7 membered heterocycloalkyl, phenyl, 5 or 6 membered heteroaryl, C₇-C₁₀ arylalkyl, C(═O)R¹⁶, C(═O)OR¹⁶, OC(═O)R¹⁶, C(═O)NR¹⁷R¹⁸, NR¹⁵C(═O)R¹⁶, NR¹⁵CO₂R¹⁶, OC(═O)NR¹⁷R¹⁸, NR¹⁵C(═S)R¹⁶, SR¹⁶; S(═O)R¹⁶; and S(═O)₂R⁶, and NR¹⁵S(═O)₂R¹⁶;

R⁴ and R⁵ at each occurrence are each independently selected from H, C₁-C₆ alkyl, C₂-C₆ alkenyl, C₂-C₆ alkynyl;

alternatively, R⁴ and R⁵, together with the carbon atom to which they are attached, form a 3-7 membered spirocyclic ring;

R¹¹ at each occurrence is independently selected from H, C₁-C₆ alkyl substituted with 0-3 R²⁰; and C₆-C₁₀ aryl substituted with 0-3 R²⁰;

R¹² and R¹³ at each occurrence are each independently selected from H, C₁-C₆ alkyl substituted with 0-3 R²⁰ and C₆-C₁₀ aryl substituted with 0-3 R²⁰; alternatively, R¹² and R¹³, together with the nitrogen to which they are attached, form a 3-7 membered heterocyclic ring substituted with 0-3 R²⁰;

R¹⁴ at each occurrence is independently selected from C₁-C₆ alkyl substituted with 0-3 R²⁰; C₆-C₁₀ aryl substituted with 0-3 R²⁰; and C₇-C₁₀ arylalkyl substituted with 0-3 R²⁰;

R¹⁵ at each occurrence is independently selected from H and C₁-C₆ alkyl;

R¹⁶ at each occurrence is independently selected from H, C₁-C₆ alkyl, and C₆-C₁₀ aryl;

R¹⁷ and R¹⁸ at each occurrence are each independently selected from H, C₁-C₆ alkyl, and C₅-C₁₀ aryl, or alternatively, R¹⁷ and R¹⁸, together with the nitrogen to which they are attached, form a 3-7 membered heterocyclic ring, wherein said 3-7 membered heterocyclic ring is substituted with 0-2 oxo groups;

R²⁰ at each occurrence is independently selected from F, Cl, Br, I, OH, OR²², OR²⁵, NR²³R²⁴, NHOH, NO₂, CN, CF₃, C₁-C₆ alkyl, C₁-C₆ alkyl-OH, C₂-C₆ alkenyl, C₂-C₆ alkynyl, C₃-C₇ cycloalkyl, 3-7 membered heterocycloalkyl, phenyl substituted by 0-1 R²⁶; 5 or 6 membered heteroaryl, C₇-C₁₀ arylalkyl, ═O, C(═O)R²², C(═O)OR²², OC(═O)R²², C(═O)NR²³R²⁴, NR²¹C(═O)R²², NR²¹CO₂R²², OC(═O)NR²³R²⁴, NR²¹C(═S)R²², SR²²; S(═O)R²²; and S(═O)₂R²²;

R²⁰A at each occurrence is independently selected from F, Cl, OH, C₁-C₄ alkoxy, CF₃, C₁-C₄ alkyl, C₁-C₄ alkyl-OH, C₂-C₄ alkenyl, C₂-C₄ alkynyl, and C₃-C₅ cycloalkyl;

R²¹ at each occurrence is independently selected from H and C₁-C₆ alkyl;

R²² at each occurrence is independently selected from H, C₁-C₆ alkyl, C₁-C₆ alkyl-OH, and C₆-C₁₀ aryl;

R²³ and R²⁴ at each occurrence are each independently selected from H, C₁-C₆ alkyl, and C₆-C₁₀ aryl, or alternatively, R²³ and R²⁴, together with the nitrogen to which they are attached, form a 3-7 membered heterocyclic ring;

R²⁵ at each occurrence is independently the residue of an amino acid after the hydroxyl group of the carboxyl group is removed;

R²⁶ at each occurrence is independently selected from H, F, Cl, Br, C₁-C₆ alkyl, and C₁-C₆ alkoxy;

x is 0, 1, 2, 3 or 4; and

q is 1 or 2.

In yet another embodiment, the modafinil intermediate compound corresponds to Formula (11):

Ar—S—Y (11);

the recovered modafinil or analog thereof corresponds to Formula (110):

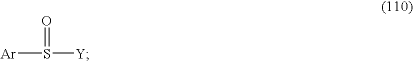

Ar is

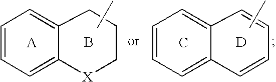

X is a bond, CH₂, O, S(O)_{y}, or NR¹⁰; rings A, C, and Dare optionally substituted with one to three groups selected from F, Cl, Br, I, OR²¹, OR²⁵, NR²³R²⁴, NHOH, NO₂, CN, CF₃, C₁-C₆ alkyl, C₂-C₆ alkenyl, C₂-C₆ alkynyl, C₃-C₇ cycloalkyl, 3-7 membered heterocycloalkyl, phenyl, 5 or 6 membered heteroaryl, arylalkyl, C(═O)R², CO₂R²¹, OC(═O)R²², C(═O)NR²³R²⁴, NR²¹C(═O)R²², NR²¹CO₂R²², OC(═O)NR²³R²⁴, NR²¹C(═S)R²², and S(O)_{y}R²²; ring B is optionally substituted with one to three groups selected from C₁-C₆ alkyl, phenyl, and 56 membered heteroaryl;

Y is (C₁-C₆ alkylene)-R¹; or (C₁-C₄ alkylene)ₘ-Z-(C₁-C₄ alkylene)ₙ-R¹; wherein said alkylene groups are optionally substituted with one to three R²⁰ groups;

Z is O, NR^{10A}, S(O)_{y}, CR²¹═CR²¹, C═C(R²¹)₂, C≡C, C₆-C₁₀ arylene, 5-10 membered heteroarylene, C₃-C₆ cycloalkylene, or 3-6 membered heterocycloalkylene; wherein said arylene, heteroarylene, cycloalkylene, and heterocycloalkylene groups are optionally substituted with one to three R²⁰ groups;

R¹ is NR¹²R¹³, NR²¹C(═O)R¹⁴, C(═O)R¹⁵, COOH, CO₂R¹⁴, OC(═O)R¹¹, C(═O)NR¹²R¹³, C(═N)NR¹²R¹³, OC(═O)NR¹²R¹³, NR²¹S(O)₂R¹¹, S(O)₂NR¹²R¹³, NR²¹C(═O)NR¹²R¹³, NR²¹S(O)₂NR¹²R¹³, or PO(OR²¹)₂;

R¹⁰ and R^{10A} are each independently selected from H, C₁-C₆ alkyl, C₆-C₁₀ aryl, C(═O)R¹⁵, and S(O)_{y}R¹⁴; wherein said alkyl and aryl groups are optionally substituted with one to three R²⁰ groups;

R¹¹ at each occurrence is independently selected from H, C₁-C₆ alkyl, and C₆-C₁₀ aryl; wherein said alkyl and aryl groups are optionally substituted with one to three R²⁰ groups;

R¹² and R¹³ at each occurrence are each independently selected from H, C₁-C₆ alkyl, and C₆-C₁₀ aryl, or R¹² and R¹³, together with the nitrogen to which they are attached, form a 3-7 membered heterocycloalkyl ring; wherein said alkyl and aryl groups and heterocycloalkyl ring are optionally substituted with one to three R²⁰ groups;

R¹⁴ at each occurrence is independently selected from C₁-C₆ alkyl, C₆-C₁₀ aryl, and arylalkyl; wherein said alkyl, aryl and arylalkyl groups are optionally substituted with one to three R²⁰ groups;

R¹⁵ at each occurrence is independently selected from C₁-C₆ alkyl, C₆-C₁₀ aryl, arylalkyl, and heteroaryl; wherein said alkyl, aryl, arylalkyl, and heteroaryl groups are optionally substituted with one to three R²⁰ groups;

R²⁰ at each occurrence is independently selected from F, Cl, Br, I, OR²¹, OR²⁵, NR²³R²⁴, NHOH, NO₂, CN, CF₃, C₁-C₆ alkyl, C₃-C₆ spirocycloalkyl, C₂-C₆ alkenyl, C₂-C₆ alkynyl, C₃-C₇ cycloalkyl, 3-7 membered heterocycloalkyl, phenyl, 5 or 6 membered heteroaryl, arylalkyl, ═O, C(═O)R²², CO₂R²¹, OC(═O)R²², C(═O)NR²³R²⁴, NR²¹C(═O)R²², NR²¹CO₂R²², OC(═O)NR²³R²⁴NR²¹C(═O)R²², NR²¹C(═S)R²², and S(O)_{y}R²²;

R²¹ at each occurrence is independently selected from H and C₁-C₆ alkyl;

R²² at each occurrence is independently selected from H, C₁-C₆ alkyl and C₆-C₁₀ aryl;

R²³ and R²⁴ at each occurrence are each independently selected from H, C₁-C₆ alkyl, and C₆-C₁₀ aryl, or R²³ and R²⁴, together with the nitrogen to which they are attached, form a 3-7 membered heterocycloalkyl ring;

R²⁵ at each occurrence is independently the residue of an amino acid after the hydroxyl group of the carboxyl group is removed;

m is 0 or 1;

n is 0 or 1;

q is 0, 1 or 2; and

y is 0, 1 or 2.

Generally speaking, the modafinil intermediate compounds and the modafinil analogs described above (i.e., the modafinil intermediate compounds corresponding to Formulae (3), (4), (5), (6), (7), (8), (9), and (11), and the modafinil analogs corresponding to Formulae (30), (40), (50), (60), (70), (80), (90), and (110)) correspond to compounds produced according to the processes described in U.S. Pat. Nos. 6,492,396, 6,670,358, and 6,919,367 to Bacon et al., and U.S. Published Patent Application Nos. 2005/0192313, 2005/0234040, 2005/20050245747, and 2005/0228040 to Bacon et al., each of which is hereby incorporated by reference herein. The processes for producing the various modafinil intermediate compounds and modafinil analogs described by Bacon et al. (and other modafinil analogs described herein) typically correspond to the general synthetic procedures illustrated in Reaction Schemes 9(a)-(f), wherein A and Y are defined as above.

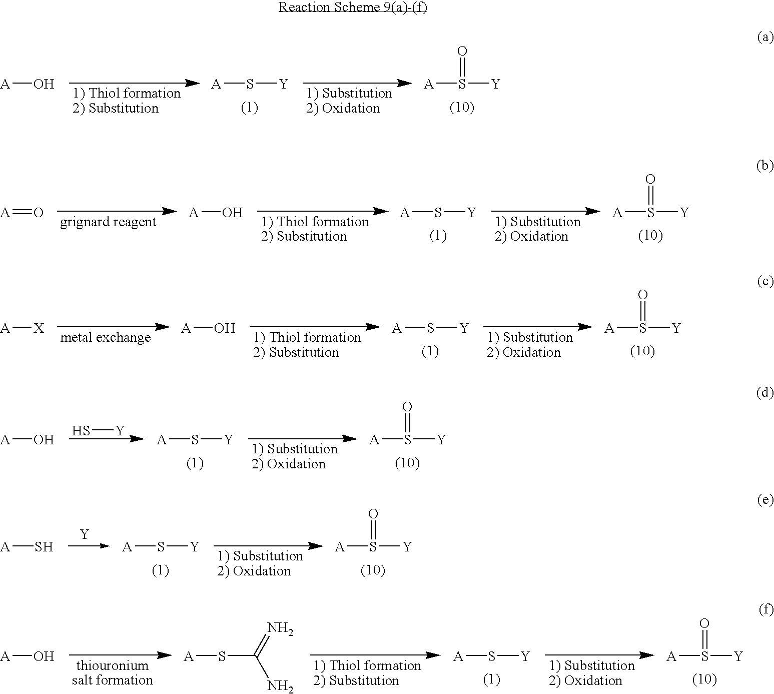

As described in detail above, the oxidation step does not necessarily need to be the last or near the last step in the synthesis process. The various intermediates may be oxidized according to the process of the present invention at any practical point in the synthesis and the oxidized compounds recovered or further derivatized to produce the desired compound.

Modafinil and analogs thereof are produced according to the process of the present invention by forming a reaction mixture including a modafinil intermediate compound described in detail above, an alcohol, and an organic acid. The modafinil intermediate compound is then oxidized with an oxidizing agent.

The ratio of alcohol to organic acid in the reaction mixture is preferably from about 1:1 to about 80:1 (by volume). More preferably, the ratio of alcohol to organic acid in the reaction mixture is from about 1:1 to about 40:1 (by volume). For example, the ratio of alcohol to organic acid in the reaction mixture may be from about 1:1 to about 5:1 (by volume), from about 1:1 to about 10:1 (by volume), from about 1:1 to about 15:1 (by volume), from about 1:1 to about 20:1 (by volume), from about 1:1 to about 25:1 (by volume), from about 1:1 to about 30:1 (by volume), from about 1:1 to about 35:1 (by volume), or from about 1:1 to about 40:1 (by volume). Still more preferably, the ratio of alcohol to organic acid in the reaction mixture is from about 1:1 to about 7:1 (by volume). For example, the ratio of alcohol to organic acid in the reaction mixture may be from about 1:1 to about 2:1 (by volume), from about 1:1 to about 3:1 (by volume), from about 1:1 to about 4:1 (by volume), from about 1:1 to about 5:1 (by volume), from about 1:1 to about 6:1 (by volume), or from about 1:1 to about 7:1 (by volume). Most preferably, the ratio of alcohol to organic acid in the reaction mixture is about 3:1 (by volume). In contrast to dissolving the modafinil intermediate compound in, for example, acetic acid alone, these particular ranges of ratios of alcohol to organic acid advantageously minimize the amount of sulfone impurity produced during the oxidation process and can produce modafinil and analogs thereof in high yield prior to recrystallization.

Any suitable linear, branched, or cyclic alcohol can be used in the process of the present invention. Suitable alcohols include, for example, methanol, ethanol, propanol, isopropanol, butanol, sec-butanol, tert-butanol, 2-methyl-1-butanol, ethylene glycol, cyclohexanol, and the like. Preferably, the alcohol is methanol.

Any suitable organic acid can be used in the process of the present invention. By way of example, the organic acid can be a carboxylic acid such as, for example, formic acid, acetic acid, propionic acid, butyric acid, oxalic acid, benzoic acid, carbonic acid, lactic acid, malic acid, tartaric acid, mandelic acid, citric acid, fumaric acid, sorbic acid, succinic acid, adipic acid, glycolic acid, glutaric acid, and the like. The organic acid can also be a sulfonic acid such as, for example, methanesulfonic acid, benzenesulfonic acid, trifluoromethenesulfonic acid, and the like. Preferably, the organic acid is a carboxylic acid such as formic or acetic acid. Most preferably, the organic acid is acetic acid.

Any suitable oxidizing agent can be used in the process of the present invention. Suitable oxidizing agents for use in the process of the present invention include, for example, O₂, K₂S₂O₈, Ca(OCl)₂, NaClO₂, NaOCl, HNO₃, NaIO₄, m-chloroperoxybenzoic acid, acylnitrates, sodium perborate, tert-butyl hypochlorite, hydrogen peroxide, t-butylhydroperoxide, alkyl- and acyl-peroxides such as benzoyl peroxide, peracetic acid, and the like. Preferably, the oxidizing agent is hydrogen peroxide. More preferably, the oxidizing agent is a solution of from about 25% (by weight) to about 55% (by weight) hydrogen peroxide in water. Still more preferably, the oxidizing agent is a solution of from about 30% (by weight) to about 50% (by weight) hydrogen peroxide in water. Most preferably, the oxidizing agent is a solution of about 30% (by weight) hydrogen peroxide in water.

The oxidizing agent is typically present in the reaction mixture at from about 0.80 to about 1.1 molar equivalents with respect to the modafinil intermediate compound. For example, the oxidizing agent may be present in the reaction mixture at from about 0.80 to about 0.85 molar equivalents with respect to the modafinil intermediate compound, from about 0.80 to about 0.90 molar equivalents with respect to the modafinil intermediate compound, from about 0.80 to about 0.95 molar equivalents with respect to the modafinil intermediate compound, from about 0.80 to about 1.0 molar equivalents with respect to the modafinil intermediate compound, or from about 0.80 to about 1.05 molar equivalents with respect to the modafinil intermediate compound.

More preferably, the oxidizing agent is present in the reaction mixture at from about 0.95 to about 1.07 molar equivalents with respect to the modafinil intermediate compound. For example, the oxidizing agent may be present in the reaction mixture at from about 0.95 to about 0.97 molar equivalents with respect to the modafinil intermediate compound, from about 0.95 to about 0.99 molar equivalents with respect to the modafinil intermediate compound, from about 0.95 to about 1.01 molar equivalents with respect to the modafinil intermediate compound, from about 0.95 to about 1.03 molar equivalents with respect to the modafinil intermediate compound, or from about 0.95 to about 1.05 molar equivalents with respect to the modafinil intermediate compound.

Most preferably, the oxidizing agent is present in the reaction mixture at from about 0.98 to about 1.07 molar equivalents with respect to the modafinil intermediate compound. For example, the oxidizing agent may be present in the reaction mixture at from about 0.98 to about 1.0 molar equivalents with respect to the modafinil intermediate compound, from about 0.98 to about 1.02 molar equivalents with respect to the modafinil intermediate compound, from about 0.98 to about 1.04 molar equivalents with respect to the modafinil intermediate compound, or from about 0.98 to about 1.06 molar equivalents with respect to the modafinil intermediate compound.

Typically, the reaction mixture is formed by mixing the alcohol, the organic acid, and the modafinil intermediate compound, with the alcohol and the organic acid being present in the ratios described above. The oxidizing agent is then charged to the reaction mixture to oxidize the modafinil intermediate compound.

While the order of the addition of the various reagents is not narrowly critical, the oxidizing agent is preferably added last and slowly to minimize overoxidation of the sulfide atom to sulfone. Preferably, the oxidizing agent is charged to the reaction mixture at a rate of from about 1 kg/minute to about 2 kg/minute. Additionally, the modafinil intermediate compound is preferably not added last, as overoxidation to sulfone is more likely to occur.

The oxidation of the modafinil intermediate compound according to the process described herein is typically performed at a reaction mixture temperature of at least room temperature. Preferably, the temperature of the reaction mixture during oxidation is less than about 70° C. More preferably, the temperature of the reaction mixture during oxidation is from about 20° C. to about 70° C. Still more preferably, the temperature of the reaction mixture during oxidation is from about 30° C. to about 65° C. Most preferably, the temperature of the reaction mixture during oxidation is about 40° C. Alternatively, the reaction mixture is not maintained at a particular temperature throughout the entire oxidation reaction. For example, the temperature can be maintained at any of the above temperatures for about 24 hours to about 48 hours, and then the reaction mixture may be allowed to cool and proceed without any such temperature maintenance.

The length of time for the oxidation reaction to achieve completion (i.e., to reach a yield plateau for the desired modafinil or analog thereof) typically depends on the temperature at which the oxidation is carried out. In general, however, the oxidation is typically allowed to proceed for about 1 hour to about 48 hours. More preferably, the oxidation is allowed to proceed for about 18 hours to about 24 hours. Most preferably, the oxidation is allowed to proceed for about 24 hours.

Once the oxidation is complete, the reaction mixture is typically cooled to about room temperature or cooler. Any excess oxidizing agent present in the reaction mixture can be optionally removed with, for example, sodium metabisulfite, sodium thiobisulfite, sodium sulfite, ferrous sulfite, and the like. If desired, from about 0.05 molar equivalents to about 0.2 molar equivalents with respect to the modafinil intermediate may be added to the reaction mixture to decompose any excess oxidizing agent present in the reaction mixture.

Upon completion of the oxidation reaction, the oxidized modafinil or analog thereof is recovered. Alternatively, if the oxidation reaction produces a modafinil-sulfoxide intermediate, the intermediate may undergo further derivatization to produce other modafinil compounds and analogs thereof, as described in Reaction Schemes 5-8 above, which may then be recovered.

Various methods for the recovery of modafinil and analogs thereof from reaction mixtures are known. Typically, the modafinil or analog thereof can be recovered from the reaction mixture by cooling, precipitating, filtering, and drying the precipitate.

The recovered modafinil or analog thereof may be optionally purified by recrystallization methods known to those of ordinary skill in the art. For example, in U.S. Pat. No. 4,177,290 to Lafon, methanol or a methanol:water mixture is used to purify modafinil by recrystallization. The use of methanol as a recrystallization solvent, however, is often relatively inefficient, or in some cases, inadequate to obtain pharmaceutically pure modafinil. Often, this is the case where several impurities are present at greater than 0.1% (by weight). Modafinil is often only mildly soluble in alcoholic solvents, even at reflux temperatures. Modafinil impurities are also relatively insoluble in alcoholic recrystallization solvents, therefore upon filtration they are only moderately reduced. Moreover, the processes of the present invention produce a highly pure recovered product prior to recrystallization, therefore a recrystallization step may not be necessary or desired.

If desired, the recovered modafinil or analog thereof may be recrystallized by mixing it with a halo-organic solvent such as, for example, dichloromethane, dichloroethane, chloroform, and the like. Typically, the halo-organic solvent is chloroform. Advantageously, modafinil and analogs thereof produced by the processes of the present invention tend to be relatively insoluble in chloroform, while the major impurities (such as, for example, modafinil acid, modafinil sulfone acid, and modafinil sulfone) are relatively soluble in chloroform.

The recovered modafinil or analog thereof/halo-organic solvent mixture tends to form a relatively viscous slurry. To reduce the viscosity, the mixture is preferably first charged with a low boiling aliphatic solvent, followed by the slow addition of the halo-organic solvent. Suitable low-boiling aliphatic solvents include, for example, pentane, hexane, octane, heptane, and the like. Preferably, the low-boiling aliphatic solvent is heptane.

The processes described herein are effective in minimizing the overoxidation of the sulfide atom to sulfone in the preparation of modafinil and analogs thereof. Generally, the overall purity of the recovered modafinil or analogs thereof (e.g., the amount of modafinil or analog thereof, sulfone impurity, and other impurities) may be determined by chromatography (e.g., HPLC at about 225 nm). Typically, not more than about 0.1% (by area as determined by HPLC) sulfone impurity is present in the recovered modafinil or analog thereof prior to recrystallization. Preferably, not more than about 0.05% (by area as determined by HPLC) sulfone impurity is present in the recovered modafinil or analog thereof prior to recrystallization; more preferably, not more than about 0.02% (by area as determined by HPLC) is present. Most preferably, the recovered modafinil or analog thereof is substantially free of the sulfone impurity. As utilized herein, “substantially free of the sulfone impurity” refers to a recovered modafinil or analogs thereof having less than about 0.05% (by area as determined by HPLC) sulfone impurity prior to recrystallization.

The processes described herein are also effective in producing highly pure modafinil and analogs thereof prior to any recrystallization of the recovered modafinil or analog thereof. Preferably, the recovered modafinil or analog thereof is greater than about 80% pure prior to recrystallization. More preferably, the recovered modafinil or analog thereof is greater than about 85% pure prior to recrystallization. Still more preferably, the recovered modafinil or analog thereof is greater than about 90% pure prior to recrystallization. Still more preferably, the recovered modafinil or analog thereof is greater than about 95% pure prior to recrystallization. Still more preferably, the recovered modafinil or analog thereof is greater than about 99% pure prior to recrystallization. Most preferably, the recovered modafinil or analog thereof is greater than about 99.5% pure prior to recrystallization.

### ABBREVIATIONS AND DEFINITIONS

The following definitions and methods are provided to better define the present invention and to guide those of ordinary skill in the art in the practice of the present invention. Unless otherwise noted, terms are to be understood according to conventional usage by those of ordinary skill in the relevant art.

As used herein, the term “alkyl” refers to a substituted or unsubstituted, branched or straight hydrocarbon chain of 1 to 8 carbon atoms, which is formed by the removal of one hydrogen atom. In certain preferred embodiments, the alkyl group contains from 1 to 6 carbon atoms. In other preferred embodiments, the alkyl group contains from 1 to 4 carbon atoms. A designation such as “C₁-C₄ alkyl” refers to an alkyl radical containing from 1 to 4 carbon atoms. Examples include methyl, ethyl, n-propyl, isopropyl, n-butyl, isobutyl, sec-butyl, t-butyl, pentyl, 2-methylpentyl, hexyl, 2-methylhexyl, 2,3-dimethylhexyl, heptyl, octyl, etc.

As used herein, the term “lower alkyl,” refers to a C₁ to C₆ saturated straight chain, branched, or cyclic hydrocarbon, which are optionally substituted. Lower alkyl groups include, but are not limited to, methyl, ethyl, n-propyl, isopropyl, n-butyl, isobutyl, t-butyl, n-pentyl, cyclopentyl, isopentyl, neopentyl, n-hexyl, isohexyl, cyclohexyl, 3-methylpentyl, 2,2-dimethylbutyl, 2,3-dimethylbutyl and the like.

As used herein, “alkenyl” refers to a substituted or unsubstituted, straight or branched hydrocarbon chain containing from 2 to 8 carbon atoms having one or more carbon-carbon double bonds which may occur in any stable point along the chain, and which is formed by removal of one hydrogen atom. A designation “C₂-C₈ alkenyl” refers to an alkenyl radical containing from 2 to 8 carbon atoms. Examples include ethenyl, propenyl, isopropenyl, 2,4-pentadienyl, etc.

As used herein, “alkynyl” refers to a substituted or unsubstituted, straight or branched hydrocarbon radical containing from 2 to 8 carbon atoms, having one or more carbon-carbon triple bonds which may occur in any stable point along the chain, and which is formed by removal of one hydrogen atom. A designation “C₂-C₈ alkynyl” refers to an alkynyl radical containing from 2 to 8 carbon atoms. Examples include ethynyl, propynyl, isopropynyl, 3,5hexadiynyl, etc.

As used herein, the term “aryl” refers to a substituted or unsubstituted, mono- or bicyclic hydrocarbon aromatic ring system having 6 to 12 ring carbon atoms. Examples include phenyl and naphthyl. Preferred aryl groups include unsubstituted or substituted phenyl and naphthyl groups. Included within the definition of “aryl” are fused ring systems, including, for example, ring systems in which an aromatic ring is fused to a cycloalkyl ring. Examples of such fused ring systems include, for example, indane, indene, and tetrahydronaphthalene.

As used herein, the terms “carbocycle”, “carbocyclic” or “carbocyclyl” refer to a substituted or unsubstituted, stable monocyclic or bicyclic hydrocarbon ring system which is saturated, partially saturated or unsaturated, and contains from 3 to 10 ring carbon atoms. Accordingly the carbocyclic group may be aromatic or non-aromatic, and includes the cycloalkyl and aryl compounds defined herein. The bonds connecting the endocyclic carbon atoms of a carbocyclic group may be single, double, triple, or part of a fused aromatic moiety.

As used herein, the term “cycloalkyl” refers to a saturated or partially saturated mono- or bicyclic alkyl ring system containing 3 to 10 carbon atoms. A designation such as “C₅-C₇ cycloalkyl” refers to a cycloalkyl radical containing from 5 to 7 ring carbon atoms. Preferred cycloalkyl groups include those containing 5 or 6 ring carbon atoms. Examples of cycloalkyl groups include such groups as cyclopropyl, cyclobutyl, cyclopentyl, cyclohexyl, cycloheptyl, cyclooctyl, pinenyl, and adamantanyl.

As used herein, the terms “heterocycle” or “heterocyclic” refer to a substituted or unsubstituted, saturated, partially unsaturated or unsaturated, stable 3 to 10 membered monocyclic or bicyclic ring wherein at least one member of the ring is a hetero atom. Accordingly the heterocyclic group may be aromatic or non-aromatic. Typically, heteroatoms include, but are not limited to, oxygen, nitrogen, sulfur, selenium, and phosphorus atoms. Preferable heteroatoms are oxygen, nitrogen and sulfur. The nitrogen and sulfur heteroatoms may be optionally oxidized, and the nitrogen may be optionally substituted in non-aromatic rings. The bonds connecting the endocyclic atoms of a heterocyclic group may be single, double, triple, or part of a fused aromatic moiety. Heterocycles are intended to include “heterocyclyl” and “heteroaryl” compounds defined herein.

As used herein, “heterocyclyl” refers to a substituted or unsubstituted, saturated, or partially unsaturated, stable 3 to 7 membered heterocyclic ring which is formed by removal of one hydrogen atom. Examples include epoxyethyl, pyrrolidyl, pyrazolidinyl, piperidyl, pyranyl, oxazolinyl, morpholino, morpholinyl, piperazinyl, etc.

Examples of heterocycles include, but are not limited to, 2-pyrrolidinyl, 2H-pyrrolyl, 4-piperidinyl, 6H-1,2,5-thiadiazinyl, 2H,6H-1,5,2-dithiazinyl, furanyl, furazanyl, imidazolidinyl, imidazolinyl, imidazolyl, isoxazolyl, morpholinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, piperazinyl, piperidinyl, pteridinyl, piperidonyl, 4-piperidinyl, purinyl, pyranyl, pyrazinyl, pyrazolidinyl, pyrazolinyl, pyrazolyl, pyridazinyl, pyridinyl, pyridyl, pyrimidinyl, pyrrolidinyl, pyrrolinyl, pyrrolyl, tetrahydrofuranyl, 6H-1,2,5-thiadiazinyl, 1,2,3-thiadiazolyl, 1,2,4-thiadiazolyl, 1,2,5-thiadiazolyl, 1,3,4-thiadiazolyl, thiazolyl, thienyl, thienothiazolyl, thienooxazolyl, thienoimidazolyl, triazinyl, 1,2,3-triazolyl, 1,2,4-triazolyl, 1,2,5-triazolyl, 1,3,4-triazolyl, and tetrazole. Suitable heterocycles are also disclosed in The Handbook of Chemistry and Physics, 76th Edition, CRC Press, Inc., 1995-1996, pages 2-25 to 2-26, the disclosure of which is hereby incorporated by reference.

Preferred heterocyclic groups formed with a nitrogen atom include, but are not limited to, pyrrolidinyl, piperidinyl, piperidino, morpholinyl, morpholino, thiomorpholino, N-methylpiperazinyl, indolyl, isoindolyl, imidazole, imidazoline, oxazoline, oxazole, triazole, thiazoline, thiazole, isothiazole, thiadiazoles, triazines, isoxazole, oxindole, indoxyl, pyrazole, pyrazolone, pyrimidine, pyrazine, quinoline, iosquinoline, and tetrazole groups.

Preferred heterocyclic groups formed with an oxygen atom include, but are not limited to, furan, tetrahydrofuran, pyran, benzofurans, isobenzofurans, and tetrahydropyran groups. Preferred heterocyclic groups formed with a sulfur atom include, but are not limited to, thiophene, thianaphthene, tetrahydrothiophene, tetrahydrothiapyran, and benzothiophenes.

Preferred aromatic heterocyclic groups include, but are not limited to, pyridyl, pyrimidyl, pyrrolyl, furyl, thienyl, imidazolyl, triazolyl, tetrazolyl, quinolyl, isoquinolyl, benzoimidazolyl, thiazolyl, pyrazolyl, and benzothiazolyl groups.

As used herein, the term “heterocycloalkyl” refers to a cycloalkyl group in which one or more ring carbon atoms are replaced by at least one hetero atom such as —O—, —N—, or —S—. Examples of heterocycloalkyl groups include pyrrolidinyl, pyrrolinyl, imidazolidinyl, imidazolinyl, pirazolidinyl, pirazolinyl, pyrazalinyl, piperidyl, piperazinyl, morpholinyl, thiomorpholinyl, tetrahydrofuranyl, dithiolyl, oxathiolyl, dioxazolyl, oxathiazolyl, pyranyl, oxazinyl, oxathiazinyl, and oxadiazinyl.

As used herein, the term “heteroaryl” refers to an aromatic group containing 5 to 10 ring carbon atoms in which one or more ring carbon atoms are replaced by at least one hetero atom such as —O—, —N—, or —S—. Examples of heteroaryl groups include pyrrolyl, furyl, thienyl, pyrazolyl, imidazolyl, thiazolyl, isothiazolyl, isoxazolyl, oxazolyl, oxathiolyl, oxadiazolyl, triazolyl, oxatriazolyl, furazanyl, tetrazolyl, pyridyl, pyrazinyl, pyrimidinyl, pyridazinyl, triazinyl, picolinyl, indolyl, isoindolyl, indazolyl, benzofiliranyl, isobenzofuranyl, purinyl, quinazolinyl, quinolyl, isoquinolyl, benzoimidazolyl, benzothiazolyl, benzothiophenyl, thianaphthenyl, benzoxazolyl, benzisoxazolyl, cinnolinyl, phthalazinyl, naphthyridinyl, and quinoxalinyl. Included within the definition of “heteroaryl” are fused ring systems, including, for example, ring systems in which an aromatic ring is fused to a heterocycloalkyl ring. Examples of such fused ring systems include, for example, phthalamide, phthalic anhydride, indoline, isoindoline, tetrahydroisoquinoline, chroman, isochroman, chromene, and isochromene.

As used herein, the term “arylalkyl” refers to an alkyl group that is substituted with an aryl group. A designation “C₇-C₁₀ arylalkyl” refers to an alkyl group that is substituted with an aryl group with the combination thereof containing from 7 to 10 carbon atoms. Examples of arylalkyl groups include, but are not limited to, benzyl, phenethyl, phenpropyl, phenbutyl, diphenylmethyl, triphenylmethyl, diphenylethyl, naphthylmethyl, etc. Preferred examples of arylalkyl groups include, but are not limited to, benzyl and phenethyl.

As used herein, the term “spirocycloalkyl” refers to a cycloalkyl group bonded to a carbon chain or carbon ring moiety by a carbon atom common to the cycloalkyl group and the carbon chain or carbon ring moiety. For example, a C₃ alkyl group substituted with an R group wherein the R group is spirocycloalkyl containing 5 carbon atoms refers to:

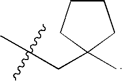

As used herein, the term “substituted” refers to replacement of one or more hydrogen atoms on an indicated group with a selected group referred to herein as a “substituent”, provided that the substituted atom's valency is not exceeded, and that the substitution results in a stable compound. A substituted group has 1 to 5, preferably 1 to 3, and more preferably 1, independently selected substituents. Preferred substituents include, but are not limited to F, Cl, Br, I, OH, OR, NH₂, NR₂, NHOH, NO₂, CN, CF₃, CF₂CF₃, C₁-C₆ alkyl, C₂-C₆ alkenyl, C₂-C₆ alkynyl, C₁-C₆ alkoxy, C₃-C₇ cycloalkyl, heterocyclyl, C₈-C₁₀ aryl, heteroaryl, arylalkyl, C(═O)R, COOH, CO₂R, O—C(═O)R, C(═O)NRR′, NRC(═O)R′, NRCO₂R′, OC(═O)NRR′, —NRC(═O)NRR′, —NRC(═S)NRR′, and —SO₂NRR′, wherein R and R′ are each independently hydrogen, C₁-C₆ alkyl, or C₆-C₁₀ aryl.

As used herein, the term “alkylene” refers to a substituted or unsubstituted, branched or straight chained hydrocarbon of 1 to 8 carbon atoms, which is formed by the removal of two hydrogen atoms. A designation such as “C₁-C₄ alkylene” refers to an alkylene radical containing from 1 to 4 carbon atoms. Examples include methylene (—CH₂—), propylidene (CH₃CH₂CH═), 1,2-ethandiyl (—CH₂CH₂—), etc.

As used herein, the term “heterocyclylene” refers to a substituted or unsubstituted, saturated, or partially unsaturated, stable 3 to 7 membered heterocyclic ring, which is formed by removal of two hydrogen atoms. Examples include epoxyethylene, pyrrolidylene, pyrrolidylidene, pyrazolidinylene, piperidylene, pyranylene, morpholinylidene, etc.

As used herein, the term “arylene” refers to a substituted or unsubstituted aromatic carbocyclic ring containing from 6 to 10 carbon atoms, which is formed by removal of two hydrogen atoms. Examples include phenylene (—C₆H₄—), naphthylene (—C₁₀H₆—), etc. The “phenylene” group has the following structure:

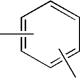

As used herein, the term “heteroarylene” refers to a substituted or unsubstituted 5 to 10 membered aromatic heterocyclic ring formed by removal of two hydrogen atoms. Examples include the heteroarylene groups which correspond to the respective heteroaryl compounds described above, and in particular, include thienylene (—C₄H₂S—), pyridylene (—C₃H₃N—), pyrimidinylene (—C₃H₂N₂—), quinolinylene (—C₉H₅N—), thiazolylene (—C₃HNS—), etc. The “thienylene” group has the following structure:

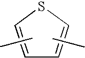

The “pyridylene” group has the following structure:

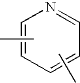

As used herein, the term “alkoxy” refers to an oxygen radical substituted with an alkyl group. Preferably, the alkoxy group contains from 1 to 6 carbon atoms. A designation such as “C₁-C₄ alkoxy” refers to an alkoxy containing from 1 to 4 carbon atoms. Examples include methoxy, ethoxy, n-propoxy, isopropoxy, n-butoxy, isobutoxy, sec-butoxy, t-butoxy, etc.

As used herein, “C₅-C₇ monosaccharide” refers to simple sugars of the formula (CH₂O)ₙ wherein n=5-7. The monosaccharides can be straight-chain or ring systems, and can include a saccharose unit of the formula —CH(OH)—C(═O)—. Examples include erythrose, threose, ribose, arabinose, xylose, lyxose, allose, altrose, glucose, mannose, gulose, idose, galactose, talose, erythulose, ribulose, xyulose, psicose, fructose, sorbose, tagatose, erythropentulose, threopentulose, glycerotetrulose, glucopyranose, fructofuranose, etc.

As used herein, the term “amino acid” refers to a molecule containing both an amino group and a carboxyl group. Embodiments of amino acids include α-amino, β-amino, γ-amino acids. The α-amino acids have a general formula HOOC—CH(side chain)-NH₂. In certain embodiments, substituent groups for the compounds of the present invention include the residue of an amino acid after removal of the hydroxyl moiety of the carboxyl group thereof; i.e., groups of formula —C(═O)CH(NH₂)-(side chain). The amino acids can be in their D, L or racemic configurations. Amino acids include naturally-occurring and non-naturally occurring moieties. The naturally-occurring amino acids include the standard 20 α-amino acids found in proteins, such as glycine, serine, tyrosine, proline, histidine, glutamine, etc. Naturally-occurring amino acids can also include non-α-amino acids (such as β-alanine, γ-aminobutyric acid, homocysteine, etc.), rare (such as 4-hydroxyproline, 5-hydroxylysine, 3-methylhistidine, etc.) and non-protein (such as citrulline, ornithine, canavanine, etc.) amino acids. Non-naturally occurring amino acids are well-known in the art, and include analogs of natural amino acids. See Lehninger, A. L. Biochemistry, 2^{nd} ed.; Worth Publishers: New York, 1975; 71-77. Non-naturally occurring amino acids also include α-amino acids wherein the side chains are replaced with synthetic derivatives. Representative side chains of naturally occurring and non-naturally occurring α-amino acids are shown below in Table A.

TABLE A
REPRESENTATIVE AMINO ACID
SIDE CHAINS

CH₃—
HO—CH₂—
C₆H₅—CH₂—
HO—C₆H₄—CH₂—

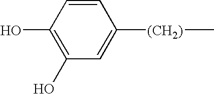

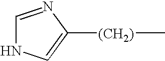

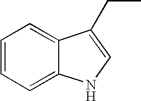

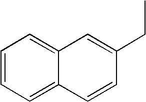

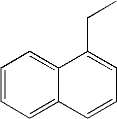

HS—CH₂—
HO₂C—CH(NH₂)—CH₂—S—S—CH₂—
CH₃—CH₂—
CH₃—S—CH₂—CH₂—
CH₃—CH₂—S—CH₂—CH₂—
HO—CH₂—CH₂—

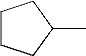

CH₃—CH(OH)—
HO₂C—CH₂—NHC(═O)—CH₂—
HO₂C—CH₂—CH₂—
NH₂C(═O)—CH₂—CH₂—
(CH₃)₂—CH—
(CH₃)₂—CH—CH₂—
CH₃—CH₂—CH₂—
H₂N—CH₂—CH₂—CH₂—
H₂N—C(═NH)—NH—CH₂—CH₂—CH₂—
H₂N—C(═O)—NH—CH₂—CH₂—CH₂—
CH₃—CH₂—CH(CH₃)—
CH₃—CH₂—CH₂—CH₂—
H₂N—CH₂—CH₂—CH₂—CH₂—

Having described the invention in detail, it will be apparent that modifications and variations are possible without departing the scope of the invention defined in the appended claims. Furthermore, it should be appreciated that all examples in the present disclosure are provided as non-limiting examples.

### EXAMPLE 1

In this Example, the modafinil intermediate compound benzhydrylthioacetamide was oxidized to produce modafinil according to the processes described herein using various ratios of alcohol to organic acid and various reaction mixture temperatures.

First, benzhydrylthioacetamide (10 g; MW=257.35; 1.0 eq.), methanol, and acetic acid were charged to a 250 mL flask. Hydrogen peroxide (4.3 mL; 1.05 eq.) was then charged to the resulting mixture over the course of about 5 minutes. The reaction was allowed to proceed for about 24 hours, with samples periodically taken for HLPC analysis. Several different trials using particular ratios of methanol and acetic acid at particular temperatures were performed. Results are illustrated in Tables 1-7, below.

TABLE 1
20 mL methanol/20 mL acetic acid; 40° C.

| | | Benzhydrylthioacetamide | |
|---|---|---|---|
| Time (hr.) | Modafinil (%) | (starting material) (%) | Sulfone (%) |
| 1 | 68 | 29.37 | 0 |
| 2 | 82.16 | 15.29 | 0 |
| 4 | 90.88 | 6.37 | 0.12 |
| 6 | 94.1 | 3.14 | 0.17 |
| 22.5 | 94.73 | 0.49 | 1.53 |

TABLE 2
30 mL methanol/10 mL acetic acid; 40° C.

| | | Benzhydrylthioacetamide | |
|---|---|---|---|
| Time (hr.) | Modafinil (%) | (starting material) (%) | Sulfone (%) |
| 0.75 | 43.07 | 53.88 | 0 |
| 1.5 | 58.5 | 38.79 | 0 |
| 4 | 77.48 | 19.9 | 0 |
| 6 | 83.99 | 13.21 | 0.04 |
| 23 | 95.98 | 1.06 | 0.22 |

TABLE 3
35 mL methanol/5 mL acetic acid; 40° C.

| | | Benzhydrylthioacetamide | |
|---|---|---|---|
| Time (hr.) | Modafinil (%) | (starting material) (%) | Sulfone (%) |
| 1 | 33.4 | 63.69 | 0 |
| 2 | 48.31 | 48.98 | 0 |
| 4.25 | 64.52 | 32.61 | 0 |
| 6 | 72.18 | 25.02 | 0 |
| 7.5 | 74.61 | 22.44 | 0 |
| 24 | 89.38 | 6.45 | 0.17 |

TABLE 4
39 mL methanol/1 mL acetic acid; 40° C.

| | | Benzhydrylthioacetamide | |
|---|---|---|---|
| Time (hr.) | Modafinil (%) | (starting material) (%) | Sulfone (%) |
| 1 | 21.87 | 74.16 | 0 |
| 3 | 40.47 | 56.52 | 0 |
| 5 | 52.49 | 43.94 | 0 |
| 7 | 60.81 | 35.63 | 0.04 |
| 23.5 | 89.6 | 7.75 | 0.1 |
| 28.75 | 88.94 | 6.84 | 0.13 |

TABLE 5
30 mL methanol/10 mL acetic acid; 65° C.

| | | Benzhydrylthioacetamide | |
|---|---|---|---|
| Time (hr.) | Modafinil (%) | (starting material) (%) | Sulfone (%) |
| 1 | 84.04 | 17.91 | 0.14 |
| 2 | 90.15 | 9.07 | 0.21 |
| 4 | 92.25 | 6.24 | 0.27 |
| 23 | 93.48 | 3.64 | 0.86 |

TABLE 6
39 mL methanol/1 mL acetic acid; 65° C.

| | | Benzhydrylthioacetamide | |
|---|---|---|---|
| Time (hr.) | Modafinil (%) | (starting material) (%) | Sulfone (%) |
| 1 | 47.67 | 52.33 | 0 |
| 2 | 69.61 | 30.12 | 0.08 |
| 3 | 78.84 | 20.68 | 0.12 |
| 5 | 86.76 | 12.7 | 0.2 |
| 6.5 | 89.72 | 9.57 | 0.24 |
| 23.5 | 96.52 | 1.68 | 0.7 |

TABLE 7
39.5 mL methanol/0.5 mL acetic acid; 65° C.

| | | Benzhydrylthioacetamide | |
|---|---|---|---|
| Time (hr.) | Modafinil (%) | (starting material) (%) | Sulfone (%) |
| 1 | 57.23 | 42.66 | 0.03 |
| 2 | 71.2 | 28.56 | 0.08 |
| 3 | 77.97 | 21.62 | 0.11 |
| 5.3 | 86.82 | 12.51 | 0.19 |
| 6.5 | 90.61 | 8.44 | 0.26 |
| 23 | 96.26 | 1.88 | 0.61 |

As illustrated in Tables 1-7 above, the processes of the present invention are effective in producing modafinil at high yield and with relatively low sulfone impurity content. Specifically, as illustrated in Table 2, a reaction mixture comprising 30 mL of methanol and 10 mL acetic acid (i.e., methanol and acetic acid are present in the reaction mixture at a ratio of about 3:1) with the oxidation reaction proceeding at 40° C. is particularly effective, producing modafinil at about 96% yield with a sulfone impurity content of about 0.22%.

### EXAMPLE 2

In this Example, the modafinil intermediate benzhydrylthioacetamide was oxidized on a commercial scale to produce modafinil according to the processes described herein.

First, benzhydrylthioacetamide (100 g; MW=257.35, 1.0 eq.) was charged to a reaction chamber. The reaction chamber was purged with about 5 psig N₂ and vented through chemical scrubber. Approximately 155 kg of methanol (1.50-1.67 kg/kg benzhydrylthioacetamide) was then charged to the reaction chamber. The temperature of the reaction chamber was adjusted to about 30° C.-40° C. and the resulting mixture was agitated at about 70-90 RPM.

Next, approximately 0.70 kg of acetic acid (0.68-0.72 kg/kg benzhydrylthioacetamide) was charged to the reaction chamber. The resulting mixture was then stirred for about 15 minutes, and the temperature was maintained at about 30° C.-40° C.

To the benzhydrylthioacetamide/methanol/acetic acid mixture was then added approximately 0.472 kg of 30% hydrogen peroxide (0.448-0.496 kg/kg benzhydrylthioacetamide) at a rate of about 1-2 kg/min. The resulting mixture was then heated to and maintained at about 38° C.-43° C. and stirred for about 24 hours.

After about 24 hours, the reaction mixture was cooled to about 20° C.-30° C. and the reaction chamber was pressurized to about 3-7 psig with N₂ and vented through a chemical scrubber. The reaction mixture was further cooled to about 0° C.-5° C. and stirred for about 2 hours. The reaction mixture was then charged to an N₂-purged centrifuge (<7% O₂ content). The centrifuge was cycled on low speed until the centrifuge basket was less than ¾ full with the crude modafinil product (˜15 minutes). The centrifuge load was washed with about 113 liters of cool methanol, and the crude modafinil cake was deliquored at high speed centrifugation for about 15-30 minutes.

The white- to off-white crude modafinil product (˜85.2 kg) was then loaded onto a Teflon®-lined tray and dried at about 60° C.-70° C. for at least about 6 hours (6-24 hours). After drying, a 5-10 gram sample was analyzed by HPLC. The results are illustrated in Table 8, below:

TABLE 8

| | % area as determined by chromatography (HPLC) | | |
|---|---|---|---|
| Results | TRIAL 1 | TRIAL 2 | TRIAL 3 |
| Modafinil | 99.74 | 99.69 | 99.7 |
| Modafinil acid | 0.05 | 0.06 | 0 |
| Modafinil sulfone | 0 | 0.04 | 0 |
| Benzhydrylthio- | 0.17 | 0.19 | 0.28 |
| acetamide | | | |

As illustrated in Examples 1 and 2, the processes of the present invention are effective in producing modafinil at high yield with relatively low sulfone impurity content prior to recrystallization.

1-52. (canceled)

53. A process for the preparation of modafinil or analogs thereof, the process comprising:
oxidizing a modafinil intermediate compound in a reaction mixture comprising an alcohol, an organic acid, and an oxidizing agent; and
recovering modafinil or analogs thereof from the reaction mixture; wherein
the ratio of alcohol to organic acid in the reaction mixture is from about 1:1 to about 80:1 (by volume);
the modafinil intermediate compound corresponds to Formula (1):
A-S—Y (1);
the recovered modafinil or analog thereof corresponds to Formula (10): 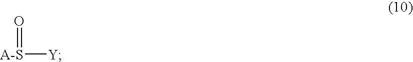
A is substituted alkyl, substituted aryl, substituted heteroaryl, or a substituted or unsubstituted tricyclic ring; and
Y is hydrocarbyl or substituted hydrocarbyl.

54. The process as set forth in claim 53 wherein the modafinil intermediate compound corresponds to Formula (2): 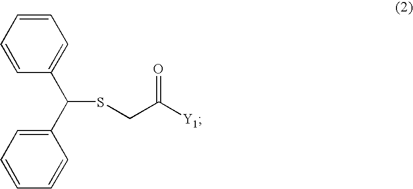
the recovered modafinil or analog thereof corresponds to Formula (20): 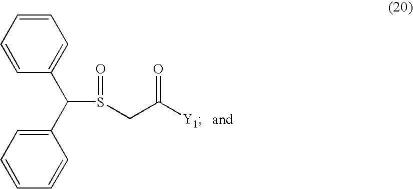
Y₁ is hydrocarbyl, hydroxy, halo, alkoxy, or amino.

55. The process as set forth in claim 53 wherein the modafinil intermediate compound corresponds to Formula (2A): 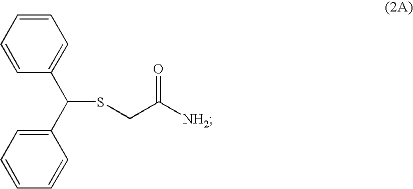
the recovered modafinil or analog thereof corresponds to Formula (200);

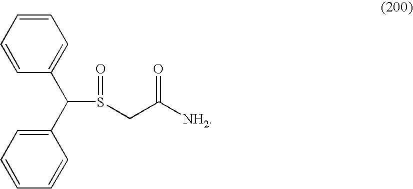

56. The process as set forth in claim 53 wherein the modafinil intermediate compound corresponds to Formula (3): 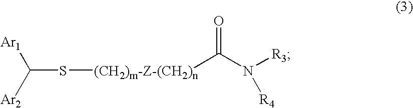
the recovered modafinil or analog thereof corresponds to Formula (30): 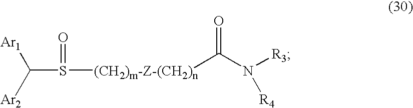
Ar₁ and Ar₂ are each independently selected from C₆-C₁₀ aryl or heteroaryl; wherein each of Ar₁ or Ar₂ may be independently optionally substituted with 1-3 substituents independently selected from;
a) H, C₆-C₁₀ aryl, heteroaryl, F, Cl, Br, I, —CN, —CF₃, —NO₂, —OH, —OR₇, —O(CH₂)ₚNR₉R₁₀, —OC(═O)R₇, —OC(═O)NR₉R₁₀, —O(CH₂)ₚOR₈, —CH₂OR₈, —NR₉R₁₀, —NR₈S(═O)₂R₇, —NR₈C(═O)R₇, or —NR₈C(═S)R₇;
b) —CH₂OR₁₁;
c) —NR₈C(═O)NR₉R₁₀, —NR₈C(═S)NR₉R₁₀, —CO₂R₁₂, —C(═O)R₁₃, —C(═O)NR₉R₁₀, —C(═S)NR₉R₁₀, —CH═NOR₁₂, —CH═NR₇, —(CH₂)ₚNR₉R₁₀, —(CH₂)ₚNHR₁₁, —CH═NNR₁₂R_{12A}, —C(═NR₈)NR_{8A}R_{8B}, —NR₈C(═NH)R_{8A}, —NR₈C(═NH)NR_{8A}R_{8B}, 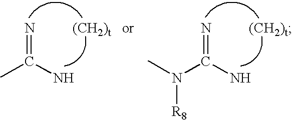
d) —S(O)_{y}R₇, —(CH₂)ₚS(O)_{y}R₇, —CH₂S(O)_{y}R₇; and
e) C₁-C₈ alkyl, C₂-C₈ alkenyl, or C₂-C₈ alkynyl, where:
1) each alkyl, alkenyl, or alkynyl group is unsubstituted; or
2) each alkyl, alkenyl or alkynyl group is independently substituted with 1 to 3 groups independently selected from C₆-C₁₀ aryl, heteroaryl, F, Cl, Br, I, CF₃, —CN, —NO₂, —OH, —OR₇, —CH₂OR₈, —NR₉R₁₀, —O—(CH₂)ₚ—OH, —S—(CH₂)ₚ—OH, —X₁(CH₂)ₚOR₇, —X₁(CH₂)ₚNR₉R₁₀, —X₁(CH₂)ₚC(═O)NR₉R₁₀, —X₁(CH₂)ₚC(═S)NR₉R₁₀, —X₁(CH₂)ₚOC(═O)NR₉R₁₀, —X₁(CH₂)ₚCO₂R₈, —X₁ (CH₂)ₚS(O)_{y}R₇, —X₁S(CH₂)ₚNR₈C(═O)NR₉R₁₀, —C(═O)R₁₃, —CO₂R₁₂, —OC(═O)R₇, —C(—O)NR₉R₁₀, —OC(═O)NR₁₂R_{12A}, O-tetrahydropyranyl, —C(═S)NR₉R₁₀, —CHNNR₁₂R_{12A}, —CHNOR₁₂, —CHNR₇, —CH═NNHCH(N═NH)NH₂, —NR₈CO₂R₇, —NR₈C(═O)NR₉R₁₀, —NR₈C(═S)NR₉R₁₀, —NHC(═NH)NH₂, —NR₈C(═O)R₇, —NR₈C(═S)R₇, —NR₈S(═O)₂R₇, —S(O)_{y}R₇, —S(═O)₂NR₁₂R_{12A}, —P(═O)(OR₈)₂, —OR₁₁, and a C₅-C₇ monosaccharide where each hydroxyl group of the monosaccharide is independently either unsubstituted or is replaced by H, C₁-C₄ alkyl, C₁-C₄ alkoxy, or —O—C(═O)R₇;
X₁ is —O—, —S—, or —N(R₈)—;
Z is selected from C₁-C₄ alkylene, —C(R₁)(R₂)—, C₆-C₁₀ arylene, heteroarylene, C₃-C₈ cycloalkylene, heterocyclylene, —O—, —N(R₈)—, —S(O)_{y}, —CR_{9A}═CR_{8B}—, —CH═CH—CH(R₈)—, —CH(R₈)—CH═CH—, or —C≡C—;
R₁, R₂, R₃ and R₄ are each independently selected from H, C₁-C₆ alkyl, —OH, and —CH(R₆)—CONR_{8A}R_{8B}; or R₃ and R₄, together with the nitrogen to which they are attached, form a 3-7 member heterocyclyl ring;
R₆ is H, C₁-C₄ alkyl, or the side chain of an α-amino acid; R₇ is C₁-C₆ alkyl, C₆-C₁₀ aryl, or heteroaryl;
R₈, R_{8A} and R_{8B} are each independently H, C₁-C₄ alkyl, or C₆-C₁₀ aryl;
R₉ and R₁₀ are each independently selected from H, C₁-C₄ alkyl, and C₆-C₁₀ aryl; or R₉ and R₁₀ together with the nitrogen to which they are attached, form a 3-7 member heterocyclyl ring;
R₁₁ is the residue of an amino acid after the hydroxyl group of the carboxyl group is removed;
R₁₂ and R_{12A} are each independently selected from H, C₁-C₆ alkyl, cycloalkyl, C₆-C₁₀ aryl, and heteroaryl; or R₁₂ and R_{12A}, together with the nitrogen to which they are attached, form a 5-7 member heterocyclyl ring;
R₁₃ is H, C₁-C₆ alkyl, cycloalkyl, C₆-C₁₀ aryl, heteroaryl, —C(═O)R₇, —C(═O)NR₉R₁₀, or —C(═S)NR₉R₁₀;
m is 0, 1, 2 or 3;
n is 0, 1, 2 or 3;
p is 1, 2, 3 or 4;
t is 2, 3 or 4; and
y is 0, 1 or 2.

57. The process as set forth in claim 53 wherein the modafinil intermediate compound corresponds to Formula (4): 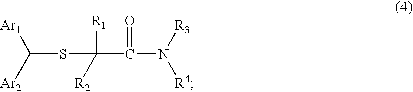
the recovered modafinil or analog thereof corresponds to Formula (40): 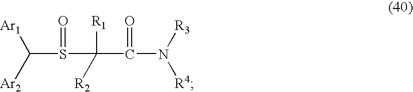
Ar₁ and Ar₂ are each independently selected from thiophene, isothiazole, phenyl, pyridyl, oxazole, isoxazole, thiazole, imidazole, and other five or six membered heterocycles comprising 1-3 atoms of —N—, —O—, or —S—;
R₁, R₂, R₃ and R₄ are each independently selected from H, lower alkyl, —OH, —CH(R₆)—CONR_{6A}R_{6B}, or any of R₁, R₂, R₃ and R₄ can be taken together to form a 3-7 member carbocyclic or heterocyclic ring; and
each of Ar₁ or Ar₂ may be independently optionally substituted with one or more substituents independently selected from:
a) H, aryl, heterocyclyl, F, Cl, Br, I, —CN, —CF₃, —NO₂, —OH, —OR₇, —O(CH₂)ₚNR₉R₁₀, —OC(═O)R₇, —OC(═O)NR₉R₁₀, —O(CH₂)ₚOR₈, —CH₂OR₈, —NR₉R₁₀, —NR₈S(═O)₂R₇, —NR₈C(═O)R₇, or —NR₈C(═S)R₇;
b) —CH₂OR₁₁, where R₁₁ is the residue of an amino acid after the hydroxyl group of the carboxyl group is removed;
c) —NR₈C(═O)NR₉R₁₀, —NR₈C(═S)NR₉R10, —CO₂R₁₂, —C(═O)R₁₂, —C(═O)NR₉R₁₀, —C(═S)NR₉R₁₀, —CH═NOR₁₂, —CH═NR₇, —(CH₂)ₚNR₉R₁₀, —(CH₂)ₚNHR₁₁, or —CH═NNR₁₂R_{12A}, where R₁₂ and R_{12A} are each independently selected from H, alkyl of 1 to 4 carbons, —OH, alkoxy of 1 to 4 carbons, —OC(═O)R₇, —OC(═O)NR₉R₁₀, —OC(═S)NR₉R₁₀, —O(CH₂)ₚNR₉R₁₀, —O(CH₂)ₚOR₈, substituted or unsubstituted arylalkyl having from 6 to 10 carbons, and substituted or unsubstituted heterocyclylalkyl;
d) —S(O)_{y}R₁₂, —(CH₂)ₚS(O)_{y}R₇, —CH₂S(O)_{y}R₁₁ where y is 0, 1 or 2; and
e) alkyl of 1 to 8 carbons, alkenyl of 2 to 8 carbons, or alkynyl of 2 to 8 carbons, where:
1) each alkyl, alkenyl, or alkynyl group is unsubstituted; or
2) each alkyl, alkenyl or alkynyl group is substituted with 1 to 3 groups selected from aryl of 6 to 10 carbons, heterocyclyl, arylalkoxy, heterocycloalkoxy, hydroxylalkoxy, alkyloxy-alkoxy, hydroxyalkylthio, alkoxy-alkylthio, F, Cl, Br, I, —CN, —NO₂, —OH, —OR₇, —X₂(CH₂)ₚNR₉R₁₀, —X₂(CH₂)ₚC(═O)NR₉R₁₀, —X₂(CH₂)ₚC(═S)NR₉R₁₀, —X₂(CH₂)ₚOC(═O)NR₉R₁₀, —X₂(CH₂)ₚCO₂R₇, —X₂(CH₂)ₚS(O)_{y}R₇, —X₂(CH₂)ₚNR₈C(—O)NR₉R₁₀, —OC(═O)R₇, —OC(═O)NHR₁₂, O-tetrahydropyranyl, —NR₉R₁₀, —NR₈CO₂R₇, —NR₈C(═O)NR₉R₁₀, —NR₈C(═S)NR₉R₁₀, —NHC(═NH)NH₂, —NR₈C(═O)R₇, —NR₈C(═S)R₇, —NR₈S(═O)₂R₇, —S(O)_{y}R₇, —CO₂R₁₂, —C(═O)NR₉R₁₀, —C(═S)NR₉R₁₀, —C(═O)R₁₂, —CH₂OR₈, —CH═NNR₁₂R_{12A}, —CH═NOR₁₂, —CH═NR₇, —CH═NNHCH(N═NH)NH₂, —S(═O)₂NR₁₂R_{12A}, —P(═O)(OR₈)₂, —OR₁₁, and a monosaccharide of 5 to 7 carbons where each hydroxyl group of the monosaccharide is independently either unsubstituted or is replaced by H, alkyl of 1 to 4 carbons, alkylcarbonyloxy of 2 to 5 carbons, or alkoxy of 1 to 4 carbons, where X₂ is O, S, or NR₈; where
R₇ is substituted or unsubstituted alkyl, substituted or unsubstituted aryl, or substituted or unsubstituted heterocyclyl;
R₈ is H or alkyl having from 1 to 4 carbons;
p is 1, 2, 3 or 4; and where either
1) R₉ and R₁₀ are each independently H, unsubstituted alkyl of 1 to 4 carbons, or substituted alkyl; or
2) R₉ and R₁₀ together form a linking group of the formula —(CH₂)₂—X₁—(CH₂)₂—, wherein X₁ is selected from —O—, —S—, and —CH₂—.

58. The process as set forth in claim 53 wherein the modafinil intermediate compound corresponds to Formula (5): 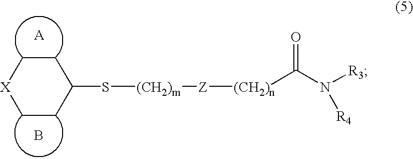
the recovered modafinil or analog thereof corresponds to Formula (50): 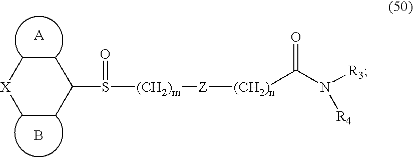
X is a bond, —CH₂CH₂—, —O—, S(O)_{y}—, —N(R₈)—, —CHN(R₈)—, —CH═CH—, —CH₂—CH═CH—, C(═O), —C(R₈)═N—, —N═C(R₈)—, —C(═O)—N(R₈)—, or —NR₈—C(═O)—;
Rings A and B, together with the carbon atoms to which they are attached, are each independently selected from;
(a) a 6-membered aromatic carbocyclic ring in which from 1 to 3 carbon atoms may be replaced by hetero atoms selected from oxygen, nitrogen and sulfur; and
b) a 5-membered aromatic carbocyclic ring in which either;
i) one carbon atom is replaced with an oxygen, nitrogen, or sulfur atom;
ii) two carbon atoms are replaced with a sulfur and a nitrogen atom, an oxygen and a nitrogen atom, or two nitrogen atoms; or
iii) three carbon atoms are replaced with three nitrogen atoms, one oxygen and two nitrogen atoms, or one sulfur and two nitrogen atoms;
wherein Ring A and Ring B may each be independently substituted with 1-3 substituents selected from:
a) H, C₆-C₁₀ aryl, heteroaryl, F, Cl, Br, I, —CN, —CF₃, —NO₂, —OH, —OR₇, —O(CH₂)ₚNR₉R₁₀, —OC(═O)R₇, —OC(═O)NR₉R₁₀, —O(CH₂)ₚOR₈, —CH₂OR₈, —NR₉R₁₀, —NR₈S(═O)₂R₇, —NR₈C(═O)R₇, or —NR₈C(═S)R₇;
b) —CH₂OR₁₁;
c) —NR₈C(═O)NR₉R₁₀, —NR₈C(═S)NR₉R₁₀, —CO₂R₁₂, —C(═O)R₁₃, —C(═O)NR₉R₁₀, —C(═S)NR₉R₁₀, —CH—NOR₁₂, —CH═NR₇, —(CH₂)ₚNR₉R₁₀, —(CH₂)ₚNHR₁₁, —CH═NNR₁₂R_{12A}, —C(═NR₈)NR_{8A}R_{8B}, —NR₈C(═NH)R_{8A}, —NR₈C(═NH)NR_{8A}R_{8B}, 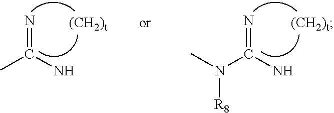
d) —S(O)_{y}R₇, —(CH₂)ₚS(O)_{y}R₇, —CH₂S(O)_{y}R₇; and
e) C₁-C₈ alkyl, C₂-C₈ alkenyl, or C₂-C₈ alkynyl, where:
1) each alkyl, alkenyl, or alkynyl group is unsubstituted; or
2) each alkyl, alkenyl or alkynyl group is independently substituted with 1 to 3 groups independently selected from C₆-C₁₀ aryl, heteroaryl, F, Cl, Br, I, CF₃, —CN, —NO₂, —OH, —OR₇, —CH₂OR₈, —NR₉R₁₀, —O—(CH₂)ₚ—OH, —S—(CH₂)ₚ—OH, —X₁(CH₂)ₚOR₇, X₁(CH₂)ₚNR₉R₁₀—X₁(CH₂)ₚC(═O)NR₉R₁₀, —X₁(CH₂)ₚC(═S)NR₉R₁₀, —X₁(CH₂)ₚOC(—O)NR₉R₁₀, —X₁(CH₂)ₚCO₂R₈, —X₁(CH₂)ₚS(O)_{y}R₇, —X₁(CH₂)ₚNR₈C(═O)NR₉R₁₀, —C(═O)R₁₃, —CO₂R₁₂, —OC(═O)R₇, —C(═O)NR₉R₁₀, —OC(═O)NR₁₂R_{12S}, O-tetrahydropyranyl, —C(═S)NR₉R₁₀, —CH═NNR₁₂R_{12A}, —CH═NOR₁₂, —CH═N₇—CH═NNHCH(N═NH)NH₂, —NR₈CO₂R₇, —NR₈C(═O)NR₉R₁₀, —NR₈C(═S)NR₉R₁₀, —NHC(═NH)NH₂, —NR₈C(═O)R₇, —NR₈C(═S)R₇, —NR₈S(═O)₂R₇, —S(O)_{y}R₇, —S(═O)₂NR₁₂R_{12A}, —P(═O)(OR₈)₂, —OR₁₁, and a C₅-C₇ monosaccharide where each hydroxyl group of the monosaccharide is independently either unsubstituted or is replaced by H, C₁-C₄ alkyl, C₁-C₄ alkoxy, or —O—C(═O)R₇;
R₃ and R₄ are each independently selected from H, C₁-C₆ alkyl, —OH, —CH(R₆)—CONR_{8A}R_{8B}, or R₃ and R₄, together with the nitrogen to which they are attached, form a 3-7 member heterocyclic ring;
R₆ is H, C₁-C₄ alkyl or the side chain of an α-amino acid;
R₇ is C₁-C₆ alkyl, C₆-C₁₀ aryl, or heteroaryl;
R₈, R_{8A} and R_{8B} are each independently H, C₁-C₄ alkyl, or C₆-C₁₀ aryl;
R₉ and R₁₀ are each independently selected from H, C₁-C₄ alkyl, and C₆-C₁₀ aryl; or R₉ and R₁₀ together with the nitrogen to which they are attached, form a 3-7 member heterocyclic ring;
R₁₁ is the residue of an amino acid after the hydroxyl group of the carboxyl group is removed;
R₁₂ and R_{12A} are each independently selected from H, C₁-C₆ alkyl, cycloalkyl, C₆-C₁₀ aryl, and heteroaryl; or R₁₂ and R_{12A}, together with the nitrogen to which they are attached, form a 5-7 member heterocyclic ring;
R₁₃ is H, C₁-C₆ alkyl, cycloalkyl, C₆-C₁₀ aryl, heteroaryl, —C(═O)R₇, —C(═O)NR₉R₁₀, or —C(═S)NR₉R₁₀;
X₁ is —O—, —S—, or —N(R₈)—;
Z is selected from C₁-C₄ alkylene, C₆-C₁₀ arylene, heteroarylene, C₃-C₈ cycloalkylene, heterocyclylene, —O—, —N(R₈)—, —S(O)_{y}, —CR_{8A}═CR_{8B}—, —CH═CH—CH(R₈)—, —CH(R₈)—CH═CH—, or —C≡C—;
m is 0, 1, 2 or 3;
n is 0, 1, 2 or 3;
p is 1, 2, 3 or 4;
q is 0, 1 or 2;
t is 2, 3 or 4; and
y is 0, 1 or 2.

59. The process as set forth in claim 53 wherein the modafinil intermediate compound corresponds to Formula (6): 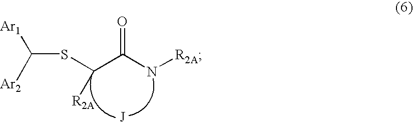
the recovered modafinil or analog thereof corresponds to Formula (60): 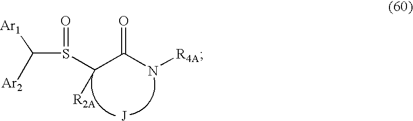
Ar₁ and Ar₂ are each independently selected from C₆-C₁₀ aryl or heteroaryl; wherein each of Ar₁ or Ar₂ may be independently optionally substituted with 1-3 substituents independently selected from;
a) H, C₆-C₁₀ aryl, heteroaryl, F, Cl, Br, I, —CN, —CF₃, —NO₂, —OH, —OR₇, —O(CH₂)ₚNR₉R₁₀, —OC(═O)R₇, —OC(═O)NR₉R₁₀, —O(CH₂)ₚOR₈, —CH₂OR₈, —NR₉R₁₀, —NR₈S(═O)₂R₇, —NR₈C(═O)R₇, or —NR₈C(═S)R₇;
b) —CH₂OR₁₁;
c) —NR₈C(═O)NR₉R₁₀, —NR₈C(═S)NR₉R₁₀, —CO₂R₁₂, —C(═O)R₁₃, —C(—O)NR₉R₁₀, —C(═S)NR₉R₁₀, —CH═NOR₁₂, —CH═NR₇, —(CH₂)ₚNR₉R₁₀, —(CH₂)ₚNHR₁₁, —CH═NNR₁₂R_{12A}, —C(═NR₈)NR_{8A}R_{8B}, —NR₈C(═NH)R_{8A}, —NR₈C(═NH)NR_{8A}R_{8B}, 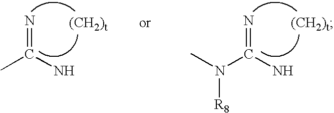
d) —S(O)_{y}R₇, —(CH₂)ₚS(O)_{y}R₇, —CH₂S(O)_{y}R₇; and
e) C₁-C₈ alkyl, C₂-C₈ alkenyl, or C₂-C₈ alkynyl, where:
1) each alkyl, alkenyl, or alkynyl group is unsubstituted; or
2) each alkyl, alkenyl or alkynyl group is independently substituted with 1 to 3 groups independently selected from C₆-C₁₀ aryl, heteroaryl, F, Cl, Br, I, CF₃, —CN, —NO₂, —OH, —OR₇, —CH₂OR₈, —NR₉R₁₀, —O—(CH₂)ₚ—OH, —S—(CH₂)ₚ—OH, —X₁(CH₂)ₚOR₇, —X₁(CH₂)ₚNR₉R₁₀, —X₁(CH₂)ₚC(═O)NR₉R₁₀, —X₁(CH₂)ₚC(═S)NR₉R₁₀, —X₁(CH₂)ₚOC(═O)NR₉R₁₀, —X₁(CH₂)ₚCO₂R₈, —X₁(CH₂)ₚS(O)_{y}R₇, —X₁S(CH₂)ₚNR₈C(═O)NR₉R₁₀, —C(═O)R₁₃, —CO₂R₁₂, —OC(═O)R₇, —C(═O)NR₉R₁₀, —OC(—O)NR₁₂R_{12A}, O-tetrahydropyranyl, —C(═S)NR₉R₁₀, —CHNNR₁₂R_{12A}, —CHNOR₁₂, —CHNR₇, —CH═NNHCH(N═NH)NH₂, —NR₈CO₂R₇, —NR₈C(═O)NR₉R₁₀, —NR₈C(═S)NR₉R₁₀, —NHC(═NH)NH₂, —NR₈C(═O)R₇, —NR₈C(═S)R₇, —NR₈S(═O)₂R₇, —S(O)_{y}R₇, —S(═O)₂NR₁₂R_{12A}, —P(═O)(OR₈)₂, —OR₁₁, and a C₅-C₇ monosaccharide where each hydroxyl group of the monosaccharide is independently either unsubstituted or is replaced by H, C₁-C₄ alkyl, C₁-C₄ alkoxy, or —O—C(═O)R₇;
X₁ is —O—, —S—, or —N(R₈)—;
J is C₂-C₄ alkylene or Q-CO—;
Q is C₁-C₃ alkylene;
R_{2A} is H, C₁-C₆ alkyl, aryl or heteroaryl;
R_{4A} is H, C₁-C₆ alkyl, aryl or heteroaryl;
R₇ is C₁-C₆ alkyl, C₆-C₁₀ aryl, or heteroaryl;
R₈, R_{8A} and R_{8B} are each independently H, C₁-C₄ alkyl, or C₆-C₁₀ aryl;
R₉ and R₁₀ are each independently selected from H, C₁-C₄ alkyl, and C₆-C₁₀ aryl; or R₉ and R₁₀ together with the nitrogen to which they are attached, form a 3-7 member heterocyclic ring;
R₁₁ is the residue of an amino acid after the hydroxyl group of the carboxyl group is removed;
R₁₂ and R_{12A} are each independently selected from H, C₁-C₆ alkyl, cycloalkyl, C₆-C₁₀ aryl, and heteroaryl; or R₁₂ and R_{12A}, together with the nitrogen to which they are attached, form a 5-7 member heterocyclic ring;
R₁₃ is H, C₁-C₆alkyl, cycloalkyl, C₆-C₁₀aryl, heteroaryl, —C(═O)R₇, —C(═O)NR₉R₁₀, or —C(═S)NR₉R₁₀;
p is 1, 2, 3 or 4;
q is 0, 1 or 2;
t is 2, 3 or 4; and
y is 0, 1 or 2.

60. The process as set forth in claim 53 wherein the modafinil intermediate compound corresponds to Formula (7): 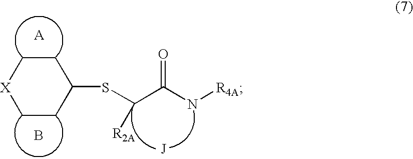
the recovered modafinil or analog thereof corresponds to Formula (70); 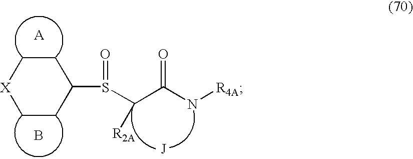
X is a bond, —CH₂CH₂—, —O—, S(O)_{y}—, —N(R₈)—, —CHN(R₈)—, —CH═CH—, —CH₂—CH═CH—, C(═O), —C(R₈)═N—, —N═C(R₈)—, —C(═O)—N(R₈)—, or —NR₈—C(═O)—;
Rings A and B, together with the carbon atoms to which they are attached, are each independently selected from:
(a) a 6-membered aromatic carbocyclic ring in which from 1 to 3 carbon atoms may be replaced by hetero atoms selected from oxygen, nitrogen and sulfur; and
b) a 5-membered aromatic carbocyclic ring in which either:
i) one carbon atom is replaced with an oxygen, nitrogen, or sulfur atom;
ii) two carbon atoms are replaced with a sulfur and a nitrogen atom, an oxygen and a nitrogen atom, or two nitrogen atoms; or
iii) three carbon atoms are replaced with three nitrogen atoms, one oxygen and two nitrogen atoms, or one sulfur and two nitrogen atoms;
wherein Ring A and Ring B may each be independently substituted with 1-3 substituents selected from:
a) H, C₆-C₁₀ aryl, heteroaryl, F, Cl, Br, I, —CN, —CF₃, —NO₂, —OH, —OR₇, —O(CH₂)ₚNR₉R₁₀, —OC(═O)R₇, —OC(═O)NR₉R₁₀, —O(CH₂)ₚOR₈, —CH₂OR₈, —NR₉R₁₀, —NR₈S(═O)₂R₇, —NR₈C(═O)R₇, or —NR₈C(═S)R₇;
b) —CH₂OR₁₁;
c) —NR₈C(═O)NR₉R₁₀, —NR₈C(═S)NR₉R₁₀, —CO₂R₁₂, —C(═O)R₁₃, —C(═O)NR₉R₁₀, —C(═S)NR₉R₁₀, —CH═NOR₁₂, —CH═NR₇, —(CH₂)ₚNR₉R₁₀, —(CH₂)ₚNHR₁₁, —CH═NNR₁₂R_{12A}, —C(—NR₈)NR_{8A}R_{8B}, —NR₈C(═NH)R_{8A}, —NR₈C(═NH)NR_{8A}R_{8B}, 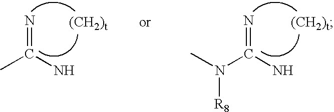
d) —S(O)_{y}R₇, —(CH₂)ₚS(O)_{y}R₇, —CH₂S(O)_{y}R₇; and
e) C₁-C₈ alkyl, C₂-C₈ alkenyl, or C₂-C₈ alkynyl, where:
1) each alkyl, alkenyl, or alkynyl group is unsubstituted; or
2) each alkyl, alkenyl or alkynyl group is independently substituted with 1 to 3 groups independently selected from C₆-C₁₀ aryl, heteroaryl, F, Cl, Br, I, CF₃, —CN, —NO₂, —OH, —OR₇, —CH₂OR₈, —NR₉R₁₀, —O—(CH₂)ₚ—OH, —S—(CH₂)ₚ—OH, —X₁(CH₂)ₚOR₇, X₁(CH₂)ₚNR₉R₁₀, —X₁(CH₂)ₚC(═O)NR₉R₁₀, —X₁(CH₂)ₚC(═S)NR₉R₁₀, —X₁(CH₂)ₚOC(═O)NR₉R₁₀, —X₁(CH₂)ₚCO₂R₈, —X₁(CH₂)ₚS(O)_{y}R₇, —X₁(CH₂)ₚNR₈C(═O)NR₉R₁₀, —C(═O)R₁₃—CO₂R₁₂, —OC(═O)R₇, —C(═O)NR₉R₁₀, —OC(═O)NR₁₂R_{12A}, O-tetrahydropyranyl, —C(═S)NR₉R₁₀, —CH—NNR₁₂R_{12A}, —CH═NOR₁₂, —CH═N₇, —CH═NNHCH(N═NH)NH₂, —NR₈CO₂R₇, —NR₈C(═O)NR₉R₁₀, —NR₈C(═S)NR₉R₁₀, —NHC(═NH)NH₂, —NR₈C(═O)R₇, —NR₈C(═S)R₇, —NR₈S(═O)₂R₇, —S(O)_{y}R₇, —S(═O)₂NR₁₂R_{12A}, —P(—O)(OR₈)₂, —OR₁₁, and a C₅-C₇ monosaccharide where each hydroxyl group of the monosaccharide is independently either unsubstituted or is replaced by H, C₁-C₄ alkyl, C₁-C₄ alkoxy, or —O—C(═O)R₇;
J is C₂-C₄ alkylene or Q-CO—;
Q is C₁-C₃ alkylene;
R_{2A} is H, C₁-C₆ alkyl, aryl or heteroaryl;
R_{4A} is H, C₁-C₆ alkyl, aryl or heteroaryl;
R₇ is C₁-C₆ alkyl, C₆-C₁₀ aryl, or heteroaryl;
R₈, R_{8A} and R_{8B} are each independently H, C₁-C₄ alkyl, or C₆-C₁₀ aryl;
R₉ and R₁₀ are each independently selected from H, C₁-C₄ alkyl, and C₆-C₁₀ aryl; or R₉ and R₁₀ together with the nitrogen to which they are attached, form a 3-7 member heterocyclic ring;
R₁₁ is the residue of an amino acid after the hydroxyl group of the carboxyl group is removed;
R₁₂ and R_{12A} are each independently selected from H, C₁-C₆ alkyl, cycloalkyl, C₆-C₁₀ aryl, and heteroaryl; or R₁₂ and R_{12A}, together with the nitrogen to which they are attached, form a 5-7 member heterocyclic ring;
R₁₃ is H, C₁-C₆ alkyl, cycloalkyl, C₆-C₁₀ aryl, heteroaryl, —C(═O)R₇, —C(═O)NR₉R₁₀, or —C(═S)NR₉R₁₀;
X is —O—, —S—, or —N(R₈)—;
p is 1, 2, 3 or 4;
q is 0, 1 or 2;
t is 2, 3 or 4; and
y is 0, 1 or 2.

61. The process as set forth in claim 53 wherein the modafinil intermediate compound corresponds to Formula (8): 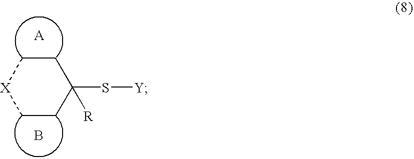
the recovered modafinil or analog thereof corresponds to Formula (80): 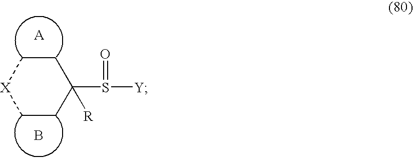
Rings A and B, together with the carbon atoms to which they are attached, are each independently selected from:
a) a 6-membered aromatic carbocyclic ring in which from 1 to 3 carbon atoms may be replaced by hetero atoms selected from oxygen, nitrogen and sulfur; and
b) a 5-membered aromatic carbocyclic ring in which either:
i) one carbon atom may be replaced with an oxygen, nitrogen, or sulfur atom;
ii) two carbon atoms may be replaced with a sulfur and a nitrogen atom, an oxygen and a nitrogen atom, or two nitrogen atoms; or
iii) three carbon atoms may be replaced with three nitrogen atoms, one oxygen and two nitrogen atoms, or one sulfur and two nitrogen atoms; wherein said rings are optionally substituted with one to three R²⁰ groups;
X is not present, is a bond, O, S(O)_{y}, NR¹⁰, C₂ alkylene, C₂₋₃ alkenylene, C(═O), C(R²¹)₂NR¹⁰, C(R²¹)═N, N═C(R²¹), C(═O)N(R¹⁰), or NR¹⁰C(═O); wherein said alkylene and alkenylene groups are optionally substituted with one to three R²⁰ groups;
R is H or C₁-C₁₀ alkyl;
Y is selected from:
a) C₁-C₆ alkylene-R¹;
b) C₁-C₆ alkylene-R²;
c) (C₁-C₄ alkylene)ₘ-Z-(C₁-C₄ alkylene)ₙ-R¹;
d) C₁-C₆ alkylene-O(CH₂)ₚOR²¹,
e) C₁-C₆ alkyl substituted with one or two OR²¹ groups; and
f) CH₂CR²¹═C(R²¹)₂;
wherein said alkyl and alkylene groups are optionally substituted with one to three R²⁰ groups;
Z is O, NR^{10A}, S(O)_{y}, CR²¹═CR²¹, C═C(R²¹)₂, C≡C, C₆-C₁₀ arylene, 5-10 membered heteroarylene, C₃-C₆ cycloalkylene, or 3-6 membered heterocycloalkylene; wherein said arylene, heteroarylene, cycloalkylene, and heterocycloalkylene groups are optionally substituted with one to three R²⁰ groups;
R¹ is selected from NR¹²R¹³, NR²¹C(═O)R¹⁴, C(═O)R¹⁵, CO₂R¹¹, OC(═O)R¹¹, C(═O)NR¹²R¹³, C(═O)NR²¹R¹⁴, C(═NR¹¹)NR¹²R¹³, NR²¹S(O)2R¹¹, S(O)₂NR¹²R¹³, NR²¹S(O)₂NR¹²R¹³, and PO(OR²¹)₂;
R² is a 5-6 membered heteroaryl, wherein said heteroaryl group is optionally substituted with one to three R²⁰ groups;
R¹⁰ and R^{10A} at each occurrence is independently selected from H, C₁-C₆ alkyl, C₆-C₁₀ aryl, C(═O)R¹⁵, and S(O)_{y}R¹⁴; wherein said alkyl and aryl groups are optionally substituted with one to three R²⁰ groups;
R¹⁴ at each occurrence is independently selected from C₁-C₆ alkyl, C₆-C₁₀ aryl, and arylalkyl; wherein said alkyl, aryl and arylalkyl groups are optionally substituted with one to three R²⁰ groups;
R¹⁵ at each occurrence is independently selected from C₁-C₆ alkyl, C₆-C₁₀ aryl, arylalkyl, and heteroaryl; wherein said alkyl, aryl, arylalkyl, and heteroaryl groups are optionally substituted with one to three R²⁰ groups;
R²⁰ at each occurrence is independently selected from F, Cl, Br, I, OR²¹, OR²⁵, NR²³R²⁴, NHOH, NO₂, CN, CF₃, C₁-C₆ alkyl, C₃-C₆ spirocycloalkyl, C₂-C₆ alkenyl, C₂-C₆ alkynyl, C₃-C₇ cycloalkyl, 3-7 membered heterocycloalkyl, phenyl, 5 or 6 membered heteroaryl, arylalkyl, ═O, C(═O)R²², CO₂R²¹, OC(═O)R²², C(═O)NR²³R²⁴, NR²¹C(═O)R²², NR²¹CO₂R²², OC(═O)NR²³R²⁴, NR²¹C(═O)R²², NR²¹C(═S)R²² and S(O)_{y}R²²;
R²¹ at each occurrence is independently selected from H and C₁-C₆ alkyl;
R²² at each occurrence is independently selected from C₁-C₆ alkyl and C₆-C₁₀ aryl;
R²³ and R²⁴ at each occurrence are each independently selected from H, C₁-C₆ alkyl, and C₆-C₁₀ aryl, or R²³ and R²⁴, together with the nitrogen to which they are attached, form a 3-7 membered heterocycloalkyl ring;
R²⁵ at each occurrence is independently the residue of an amino acid after the hydroxyl group of the carboxyl group is removed; and
y is 0, 1 or 2.

62. The process as set forth in claim 53 wherein the modafinil intermediate compound corresponds to Formula (9): 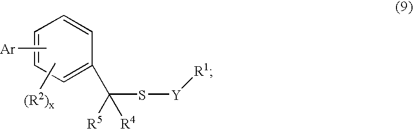
the recovered modafinil or analog thereof corresponds to Formula (90): 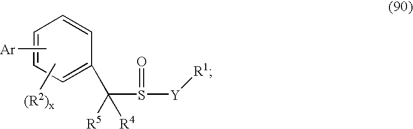
Ar is C₆-C₁₀ aryl substituted by 0-5 R₃; C₅-C₁₀ cycloalkenyl substituted by 0-5 R³; or 5 to 14 membered heteroaryl group substituted by 0-5 R³, wherein said heteroaryl group comprises one, two, or three heteroatoms selected from N, O, S or Se;
Y is C₁-C₆ alkylene substituted with 0-3 R^{20A};
R¹ is selected from H, C(═O)NR¹²R¹³, C(═N)NR¹²R¹³, OC(═O)NR¹²R¹³, NR²¹C(═O)NR¹²R¹³, NR²¹S(═O)₂NR¹²R¹³, —(C₆-C₁₀ aryl)-NR¹²R¹³ wherein said aryl is substituted with 0-3 R²⁰; NR²¹C(═O)R¹⁴, C(═O)R¹⁴, C(═O)OR¹¹, OC(═O)R¹¹, and NR²¹S(═O)₂R¹¹;
R² is selected from H, F, Cl, Br, I R¹⁶, OR²⁵, NR¹⁷R¹⁸, NHOH, NO₂, CN, CF₃, C₁-C₆ alkyl, C₂-C₆ alkenyl, C₂-C₆ alkynyl, C(═O)R¹⁶, C(═O)OR¹⁶, OC(═O)R¹⁶, C(═O)NR¹⁷R¹⁸, NR¹⁵C(═O)R¹⁶, NR¹⁵CO₂R¹⁶, OC(—O)NR¹⁷R¹⁸, NR¹⁵C(═S)R¹⁶, SR¹⁶; S(═O)R¹⁶; and S(═O)₂R¹⁶; alternatively, two R² groups may be combined to form a methylenedioxy group, an ethylenedioxy group, or a propylenedioxy group;
R³ is selected from H, F, Cl, Br, I, OR¹⁶, OCF₃, OR²⁵, NR¹⁷R¹⁸, NHOH, NO₂, CN, CF₃, CH₂OR¹⁶, C₁-C₆ alkyl, C₂-C₆ alkenyl, C₂-C₆ alkynyl, C₃-C₇ cycloalkyl, 3-7 membered heterocycloalkyl, phenyl, 5 or 6 membered heteroaryl, C₇-C₁₀ arylalkyl, C(═O)R¹⁶, C(═O)OR¹⁶, OC(═O)R¹⁶, C(═O)NR¹⁷R¹⁸, NR¹⁵C(═O)R¹⁶, NR¹⁵CO₂R¹⁶, OC(═O)NR¹⁷R¹⁸, NR¹⁵C(═S)R¹⁶, SR¹⁶; S(═O)R¹⁶; and S(═O)₂R¹⁶, and NR¹⁵S(═O)₂R¹⁶;
R⁴ and R⁵ at each occurrence are each independently selected from H, C₁-C₆ alkyl, C₂-C₆ alkenyl, C₂-C₆ alkynyl;
alternatively, R⁴ and R⁵, together with the carbon atom to which they are attached, form a 3-7 membered spirocyclic ring;
R¹¹ at each occurrence is independently selected from H, C₁-C₆ alkyl substituted with 0-3 R²⁰; and C₆-C₁₀ aryl substituted with 0-3 R²⁰;
R₁₂ and R¹² at each occurrence are each independently selected from H, C₁-C₆ alkyl substituted with 0-3 R²⁰ and C₆-C₁₀ aryl substituted with 0-3 R²⁰; alternatively, R¹² and R¹³, together with the nitrogen to which they are attached, form a 3-7 membered heterocyclic ring substituted with 0-3 R²⁰;
R¹⁴ at each occurrence is independently selected from C₁-C₆ alkyl substituted with 0-3 R²⁰; C₆-C₁₀ aryl substituted with 0-3 R²⁰; and C₇-C₁₀ arylalkyl substituted with 0-3 R²⁰;
R¹⁵ at each occurrence is independently selected from H and C₁-C₆ alkyl;
R¹⁶ at each occurrence is independently selected from H, C₁-C₆ alkyl, and C₆-C₁₀ aryl;
R¹⁷ and R¹⁸ at each occurrence are each independently selected from H, C₁-C₆ alkyl, and C₆-C₁₀ aryl, or alternatively, R¹⁷ and R¹⁸, together with the nitrogen to which they are attached, form a 3-7 membered heterocyclic ring, wherein said 3-7 membered heterocyclic ring is substituted with 0-2 oxo groups;
R²⁰ at each occurrence is independently selected from F, Cl, Br, I, OH, OR²², OR²⁵, NR²³R²⁴, NHOH, NO₂, CN, CF₃, C₁-C₆ alkyl, C₁-C₆ alkyl-OH, C₂-C₆ alkenyl, C₂-C₆ alkynyl, C₃-C₇ cycloalkyl, 3-7 membered heterocycloalkyl, phenyl substituted by 0-1 R²⁶; 5 or 6 membered heteroaryl, C₇-C₁₀ arylalkyl, ═O, C(═O)R²², C(═O)OR²², OC(═O)R²², C(═O)NR²³R²⁴, NR²¹C(═O)R²², NR²¹CO₂R²², OC(═O)NR²³R²⁴, NR²¹C(═S)R²², SR²²; S(═O)R²²; and S(═O)₂R²²;
R²⁰A at each occurrence is independently selected from F, Cl, OH, C₁-C₄ alkoxy, CF₃, C₁-C₄ alkyl, C₁-C₄ alkyl-OH, C₂-C₄ alkenyl, C₂-C₄ alkynyl, and C₃-C₅ cycloalkyl;
R²¹ at each occurrence is independently selected from H and C₁-C₆ alkyl;
R²² at each occurrence is independently selected from H, C₁-C₆ alkyl, C₁-C₆ alkyl-OH, and C₆-C₁₀ aryl;
R²³ and R²⁴ at each occurrence are each independently selected from H, C₁-C₆ alkyl, and C₆-C₁₀ aryl, or alternatively, R²³ and R²⁴, together with the nitrogen to which they are attached, form a 3-7 membered heterocyclic ring;
R²⁵ at each occurrence is independently the residue of an amino acid after the hydroxyl group of the carboxyl group is removed;
R²⁶ at each occurrence is independently selected from H, F, Cl, Br, C₁-C₆ alkyl, and C₁-C₆ alkoxy;
x is 0, 1, 2, 3 or 4; and
q is 1 or 2.

63. The process as set forth in claim 53 wherein the modafinil intermediate compound corresponds to Formula (11):
Ar—S—Y (11);
the recovered modafinil or analog thereof corresponds to Formula (110); 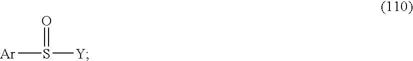
Ar is 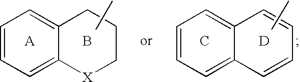
X is a bond, CH₂, O, S(O)_{y}, or NR¹⁰; rings A, C, and D are optionally substituted with one to three groups selected from F, Cl, Br, I, OR²¹, OR²⁵, NR²³R²⁴, NHOH, NO₂, CN, CF₃, C₁-C₆ alkyl, C₂-C₆ alkenyl, C₂-C₆ alkynyl, C₃-C₇ cycloalkyl, 3-7 membered heterocycloalkyl, phenyl, 5 or 6 membered heteroaryl, arylalkyl, C(═O)R²², CO₂R²¹, OC(═O)R²², C(═O)NR²³R²⁴, NR²¹C(═O)R²², NR²¹CO₂R²², OC(═O)NR²³R²⁴, NR²¹C(═S)R²², and S(O)_{y}R²²; ring B is optionally substituted with one to three groups selected from C₁-C₆ alkyl, phenyl, and 5-6 membered heteroaryl;
Y is (C₁-C₆ alkylene)-R¹; or (C₁-C₄ alkylene)ₘ-Z-(C₁-C₄ alkylene)ₙ-R¹; wherein said alkylene groups are optionally substituted with one to three R²⁰ groups;
Z is O, NR^{10A}, S(O)_{y}, CR²¹═CR²¹, C═C(R²¹)₂, C≡C, C₆-C₁₀ arylene, 5-10 membered heteroarylene, C₃-C₆ cycloalkylene, or 3-6 membered heterocycloalkylene; wherein said arylene, heteroarylene, cycloalkylene, and heterocycloalkylene groups are optionally substituted with one to three R²⁰ groups;
R¹ is NR¹²R¹³, NR²¹C(═O)R¹⁴, C(═O)R¹⁵, COOH, CO₂R¹⁴, OC(═O)R¹¹, C(═O)NR¹²R¹³, C(═N)NR¹²R¹³, OC(═O)NR¹²R¹³, NR²¹S(O)₂R¹¹, S(O)₂NR¹²R¹³, NR²¹C(═O)NR¹²R¹³, NR²¹S(O)₂NR¹²R¹³ or PO(OR²¹)₂;
R¹⁰ and R^{10A} are each independently selected from H, C₁-C₆ alkyl, C₆-C₁₀ aryl, C(═O)R¹⁵, and S(O)_{y}R¹⁴; wherein said alkyl and aryl groups are optionally substituted with one to three R²⁰ groups;
R¹¹ at each occurrence is independently selected from H, C₁-C₆ alkyl, and C₆-C₁₀ aryl; wherein said alkyl and aryl groups are optionally substituted with one to three R²⁰ groups;
R¹² and R¹³ at each occurrence are each independently selected from H, C₁-C₆ alkyl, and C₆-C₁₀ aryl, or R¹² and R¹³ together with the nitrogen to which they are attached, form a 3-7 membered heterocycloalkyl ring; wherein said alkyl and aryl groups and heterocycloalkyl ring are optionally substituted with one to three R²⁰ groups;
R¹⁴ at each occurrence is independently selected from C₁-C₆ alkyl, C₆-C₁₀ aryl, and arylalkyl; wherein said alkyl, aryl and arylalkyl groups are optionally substituted with one to three R²⁰ groups;
R¹⁵ at each occurrence is independently selected from C₁-C₆ alkyl, C₆-C₁₀ aryl, arylalkyl, and heteroaryl; wherein said alkyl, aryl, arylalkyl, and heteroaryl groups are optionally substituted with one to three R²⁰ groups;
R²⁰ at each occurrence is independently selected from F, Cl, Br, I, OR²¹, OR²⁵, NR²³R²⁴, NHOH, NO₂, CN, CF₃, C₁-C₆ alkyl, C₃-C₆ spirocycloalkyl, C₂-C₆ alkenyl, C₂-C₆ alkynyl, C₃-C₇ cycloalkyl, 3-7 membered heterocycloalkyl, phenyl, 5 or 6 membered heteroaryl, arylalkyl, ═O, C(═O)R²², CO₂R²¹, OC(═O)R²², C(—O)NR²³R²⁴, NR²¹C(═O)R²², NR²¹CO₂R²², OC(═O)NR²³R²⁴, NR²¹C(═O)R²², NR²¹C(═S)R²² and S(O)_{y}R²²;
R²¹ at each occurrence is independently selected from H and C₁-C₆ alkyl;
R²² at each occurrence is independently selected from H, C₁-C₆ alkyl and C₆-C₁₀ aryl;
R²³ and R²⁴ at each occurrence are each independently selected from H, C₁-C₆ alkyl, and C₆-C₁₀ aryl, or R²³ and R²⁴, together with the nitrogen to which they are attached, form a 3-7 membered heterocycloalkyl ring;
R²⁵ at each occurrence is independently the residue of an amino acid after the hydroxyl group of the carboxyl group is removed;
m is 0 or 1;
n is 0 or 1;
q is 0, 1 or 2; and
y is 0, 1 or 2.

64. The process as set forth in claim 63 wherein the ratio of alcohol to organic acid in the reaction mixture is from about 1:1 to about 7:1 (by volume).

65. The process as set forth in claim 64 wherein the ratio of alcohol to organic acid in the reaction mixture is about 3:1 (by volume).

66. The process as set forth in claim 65 wherein the alcohol is selected from the group consisting of linear, branched, and cyclic alcohols.

67. The process as set forth in claim 66 wherein the alcohol is selected from the group consisting of methanol, ethanol, propanol, isopropanol, butanol, sec-butanol, tert-butanol, 2-methyl-1-butanol, ethylene glycol, cyclohexanol, and combinations thereof.

68. The process as set forth in claim 67 wherein the alcohol is methanol.

69. The process as set forth in claim 68 wherein the organic acid is selected from the group consisting of carboxylic acids, sulfonic acids, and combinations thereof.

70. The process as set forth in claim 69 wherein the organic acid is selected from the group consisting of formic acid, acetic acid, propionic acid, butyric acid, oxalic acid, benzoic acid, carbonic acid, lactic acid, malic acid, tartaric acid, mandelic acid, citric acid, fumaric acid, sorbic acid, succinic acid, adipic acid, glycolic acid, glutaric acid, methanesulfonic acid, benzenesulfonic acid, trifluoromethenesulfonic acid, and combinations thereof.

71. The process as set forth in claim 70 wherein the organic acid is acetic acid.

72. The process as set forth in claim 71 wherein the oxidizing agent is selected from the group consisting of O₂, K₂S₂O₈, Ca(OCl)₂, NaClO₂, NaOCl, HNO₃, NaIO₄, m-chloroperoxybenzoic acid, acylnitrates, sodium perborate, tert-butyl hypochlorite, hydrogen peroxide, t-butylhydroperoxide, alkyl- and acyl-peroxides, benzoyl peroxide, peracetic acid, and combinations thereof.

73. The process as set forth in claim 72 wherein the oxidizing agent is hydrogen peroxide.

74. The process as set forth in claim 73 wherein the oxidizing agent is a solution of hydrogen peroxide in water.

75. The process as set forth in claim 74 wherein the oxidizing agent is a solution of from about 25% (by weight) to about 55% (by weight) hydrogen peroxide in water.

76. The process as set forth in claim 75 wherein the oxidizing agent is a solution of about 30% (by weight) hydrogen peroxide in water.

77. The process as set forth in claim 76 wherein the reaction mixture comprises from about 0.80 to about 1.1 molar equivalents of oxidizing agent with respect to the modafinil intermediate compound.

78. The process as set forth in claim 77 wherein the temperature of the reaction mixture during oxidation is at least about room temperature.

79. The process as set forth in claim 78 wherein the temperature of the reaction mixture during oxidation is from about 20° C. to about 70° C.

80. The process as set forth in claim 79 wherein the temperature of the reaction mixture during oxidation is about 40° C.

81. The process as set forth in claim 80 wherein the reaction mixture is not maintained at a particular temperature during the oxidation.

82. The process as set forth in claim 81 wherein the oxidation is allowed to proceed for about 1 hour to about 48 hours.

83. The process as set forth in claim 82 wherein the oxidation is allowed to proceed for about 24 hours.

84. The process as set forth in claim 83 further comprising recrystallizing the recovered modafinil or analogs thereof.

85. The process as set forth in claim 84 wherein the recovered modafinil or analog thereof is substantially free of sulfone impurity prior to recrystallization.

86. The process as set forth in claim 85 wherein the recovered modafinil or analog thereof has a purity of greater than about 95% prior to recrystallization.

87. The process as set forth in claim 86 wherein the recovered modafinil or analog thereof has a purity of greater than about 99% prior to recrystallization.

88. The process as set forth in claim 87 wherein the recovered modafinil or analog thereof has a purity of about 99.5% prior to recrystallization.

89. The process as set forth in claim 88 wherein the recovered modafinil or analog thereof has a purity of greater than about 99.5% prior to recrystallization.

90. The process as set forth in claim 89 wherein the recovered modafinil or analog thereof is recrystallized by mixing the modafinil or analog thereof with a low boiling aliphatic solvent and a halo-organic solvent.

91. The process as set forth in claim 90 wherein the halo-organic solvent is selected from the group consisting of dichloromethane, dichloroethane, chloroform, and combinations thereof.

92. The process as set forth in claim 91 wherein the low boiling aliphatic solvent is selected from the group consisting of pentane, hexane, octane, heptane, and combinations thereof.
